(19) United States
(12) Patent Application Publication
(10) Pub. No.: US 2023/0338284 A1
(43) Pub. Date: Oct. 26, 2023

(54) METHODS FOR THE TREATMENT OF LUNG TUMORS

(71) Applicant: GRADALIS, INC., Carrollton, TX (US)

(72) Inventors: DAVID SHANAHAN, Carrollton, TX (US); John Nemunaitis, Carrollton, TX (US); Ernest Bognar, Carrollton, TX (US)

(73) Assignee: GRADALIS, INC., Carrollton, TX (US)

(21) Appl. No.: 18/114,923

(22) Filed: Feb. 27, 2023

Related U.S. Application Data
(63) Continuation of application No. PCT/US2021/049108, filed on Sep. 3, 2021.
(60) Provisional application No. 63/076,294, filed on Sep. 9, 2020.

(51) Int. Cl. A61K 9/00; A61K 9/19; A61K 47/26; C12N 15/113; A61K 48/00

(52) U.S. Cl. CPC A61K 9/0075; A61K 9/19; A61K 47/26; C12N 15/1137; A61K 48/0075; A61K 38/193

(57) ABSTRACT

The present disclosure provides composition formulated into inhalable dosage forms for treating a tumor or cancer in the lung. The compositions can be lyophilized compositions formulated for pulmonary delivery via a device.

### CROSS-REFERENCE TO RELATED APPLICATIONS

This application is a continuation of PCT/US2021/049108, filed Sep. 3, 2021, which claims priority to U.S. Provisional Patent Application No. 63/076,294, filed Sep. 9, 2020, the contents both of which are hereby incorporated by reference in their entireties for all purposes.

### SEQUENCE LISTING

The instant application contains a Sequence Listing which has been submitted electronically is hereby incorporated by reference in its entirety. Said .xml file, created on Mar. 21, 2023, is identified as 1372942-000210US and is 23,053 bytes in size.

### BACKGROUND

Tumors in the lung can arise from lung cancer or from a metastatic event from other organs in the body. About 80% of lung cancers are Non-Small Cell Lung Cancer (NSCLC). The main subtypes of NSCLC are adenocarcinoma, squamous cell carcinoma, and large cell carcinoma. These subtypes, which start from different types of lung cells, are grouped together as NSCLC because their treatment and prognoses are often similar.

In addition, about 15% of all lung cancers are Small Cell Lung Cancer (SCLC). This type of lung cancer tends to grow and spread faster than NSCLC. About 70% of people with SCLC will have cancer that has already spread at the time they are diagnosed.

U.S. Pat. No. 9,683,237 to Rao et al. discloses compositions and methods for making and using a RNAi capable of reducing expression of two or more genes, including a first RNAi molecule that reduces the expression of a first target gene; a second RNAi molecule that reduces the expression of the first or a second target gene; and optionally a third RNAi molecule that reduces the expression of the first, the second, or a third target gene, wherein the RNAi molecules reduce the expression level of, e.g., mutated KRAS, SRC-3, EGFR, PIK3, NCOA3, or ERalphal, and can be, e.g., miRNAs, shRNAs, or bifunctional shRNAs.

U.S. Pat. No. 9,382,589 to Shanahan discloses methods for treating cancer, which comprise (a) obtaining a specimen of cancer tissue from a patient; (b) obtaining a specimen of normal tissue in the proximity of the cancer tissue from such patient; (c) extracting total protein and RNA from the cancer tissue and normal tissue; (d) obtaining a protein expression profile of the cancer tissue and normal tissue using 2D DIGE and mass spectrometry; (e) identifying proteins that are expressed in such cancer tissue at significantly different levels than in the normal tissue; (f) obtaining a gene expression profile of the cancer tissue and normal tissue using microarray technology and comparing the results thereof to the protein expression profile; (g) prioritizing over-expressed proteins by assessing the connectivity thereof to other cancer-related or stimulatory proteins; (h) designing an appropriate RNA interference expression cassette to, directly or indirectly, modulate the expression of genes encoding such prioritized proteins; (i) incorporating said cassette into an appropriate delivery vehicle; and (j) providing the patient with an effective amount of the delivery vehicle to, directly or indirectly, modify the expression (i.e., production) of such proteins.

Despite the foregoing advances in the art, there remains a need for new ways to administer plasmids that treat tumors in the lungs of a subject. The present disclosure satisfies this need and provides other advances as well.

### BRIEF SUMMARY

In one embodiment, the present disclosure provides an inhalable dosage form, comprising:
a. an expression vector comprising
   i. a first insert comprising a nucleic acid sequence encoding a Granulocyte Macrophage Colony Stimulating Factor (GM-CSF) sequence; and
   ii. a second insert comprising a nucleic acid sequence encoding at least one bi-functional short hairpin RNA (bi-shRNA) capable of hybridizing to a region of a mRNA transcript encoding furin, thereby inhibiting furin expression via RNA interference wherein the bi-shRNA incorporates cleavage dependent siRNA and cleavage independent miRNA motifs; and
b. at least one stabilizing excipient.

In certain aspects, the second insert comprises a nucleic acid sequence of SEQ ID NO:2 or SEQ ID NO:9.

In certain aspects, the expression vector is a plasmid.

In certain aspects, the inhalable dosage form comprises particles comprising the expression vector and at least one stabilizing excipient selected from the group comprising glucose, arabinose, maltose, saccharose, dextrose, lactose, sucrose, trehalose, human serum albumin (HSA), and glycine.

In certain aspects, the stabilizing excipient is trehalose.

In certain aspects, the inhalable dosage form is a particle or a plurality of particles. The particles can be from about 0.5 μm to about 10 μm in diameter.

In certain aspects, the particles are from about 1 μm to 3 μm in diameter.

In certain aspects, the expression vector is present from about 1% to about 50%, or 1% to 40%, or 1% to 30%, or 1% to 20%, or 1% to 15%, or 1% to 10%, such as about 1%, 2%, 3%, 4%, 5%, 6%, 7%, 8%, 9%, 10%, 11%, 12%, 13%, 14%, 15%, 16%, 17%, 18%, 19%, and/or 20% by weight or more of the total inhalable dosage form.

In certain aspects, the particles are lyophilized particles.

In certain aspects, the GM-CSF is a human GM-CSF sequence.

In certain aspects, the expression vector further comprises a promoter.

In certain aspects, the promoter is a cytomegalovirus (CMV) mammalian promoter.

In certain aspects, the expression vector further comprises a CMV enhancer sequence and a CMV intron sequence.

In certain aspects, the first insert and the second insert are operably linked to the promoter.

In certain aspects, the expression vector further comprises a nucleic acid sequence encoding a picornaviral 2A ribosomal skip peptide between the first and the second nucleic acid inserts.

In certain aspects, the lyophilized composition is formulated for pulmonary delivery.

In certain aspects, the lyophilized composition is formulated for pulmonary delivery via a device such as an inhaler.

In certain aspects, the device is an inhaler or a nebulizer.

In another embodiment, the present disclosure provides a method for treating a tumor in the lung of a subject, the method comprising:

administering to the subject an inhalable dosage form, comprising:
a. an expression vector comprising
   i. a first insert comprising a nucleic acid sequence encoding a Granulocyte Macrophage Colony Stimulating Factor (GM-CSF) sequence; and
   ii. a second insert comprising a nucleic acid sequence encoding at least one bi-functional short hairpin RNA (bi-shRNA) capable of hybridizing to a region of a mRNA transcript encoding furin, thereby inhibiting furin expression via RNA interference wherein the bi-shRNA incorporates cleavage dependent siRNA and cleavage independent miRNA motifs; and
b. at least one stabilizing excipient, to thereby treat the tumor.

In certain aspects, the tumor is caused by a primary lung cancer.

In certain aspects, the primary lung cancer is a member selected from the group comprising non-small cell lung cancer (NSCLC) and small cell lung cancer (SCLC).

In certain aspects, the tumor is caused by a metastatic event from a cancer originating in a different part of the subject's body.

In certain aspects, the cancer is a member selected from the group consisting of breast, pancreas, kidney, and skin cancer, although the cancer ends up or metastasizes to the lungs.

In certain aspects, the cancer is a member selected from the group comprising brain, bladder, blood, bone, breast, cervical, colorectal, gastrointestinal, endocrine, kidney, liver, lung, ovarian, pancreatic, prostate, and thyroid cancer.

These and other objects, aspects and embodiments will become more apparent when read with the detailed description and figures that follow.

### BRIEF DESCRIPTION OF THE DRAWINGS

FIG. 1A shows a schematic showing the bi-shRNA^{furin} (SEQ ID NO:2) comprising two stem-loop structures with a miR-30a backbone; wherein the first stem-loop structure has complete complementary guiding strand and passenger strand, while the second stem-loop structure has three basepair (bp) mismatches at positions 9 to 11 of the passenger strand.

FIG. 1B depicts a schematic showing a 5140 base pair (bp) SEQ ID NO:3 plasmid containing a GM-CSF gene for expression and bifunctional furin sh-RNA, along with a kanamycin cassette and CMV promoter.

FIG. 2 shows a lyophilization procedure wherein an expression vector is in solution, and can be rapidly frozen and lyophilized. After lyophilization, the lyophilized powder becomes a dry powder. A dry powder can be aerosolized in a dry powder inhaler.

FIGS. 3A-3C show cell culture results of furin concentration using ELISA to measure the concentration in three different cell lines.

FIGS. 3D-3F show percent knock down of furin by the plasmid in each cell line.

FIGS. 4A-4D show cell culture results of the expression of GM-CSF measured using an enzyme-linked lectin assay (ELLA), in 4 different cell lines.

FIGS. 5A-5D show cell culture results of the expression of TGF-β1 measured using an enzyme-linked lectin assay (ELLA), in 4 different cell lines.

### DETAILED DESCRIPTION

### I. Definition

As used herein, ranges and amounts can be expressed as “about” a particular value or range. In addition, “about” also includes the exact amount. Hence “about 5 μg” means “about 5 μg” and also “5 μg.” Generally, the term “about” includes an amount that would be expected to be within experimental error. In some aspects, “about” refers to the number or value recited, “+” or “−” 20%, 10%, or 5% (added or subtracted) of the number or value.

The terms “effective amount” or “therapeutically effective amount,” as used herein, refer to a sufficient amount of an agent or a compound being administered that will relieve to some extent one or more of the symptoms of the disease or condition being treated or prevent the onset or recurrence of the one or more symptoms of the disease or condition being treated. In some aspects, the result is reduction and/or alleviation of the signs, symptoms, or causes of a disease, or any other desired alteration of a biological system. For example, an “effective amount” for therapeutic uses is the amount of the autologous tumor cell vaccine required to provide a clinically significant decrease in disease symptoms without undue adverse side effects. In another example, an “effective amount” for therapeutic uses is the amount of the autologous tumor cell vaccine as disclosed herein required to prevent a relapse of disease symptoms without undue adverse side effects. An appropriate “effective amount” in any individual case may be determined using techniques, such as a dose escalation study. The term “therapeutically effective amount” includes, for example, a prophylactically effective amount. An “effective amount” of a compound disclosed herein, is an amount effective to achieve a desired effect or therapeutic improvement without undue adverse side effects. It is understood that, in some aspects, “an effective amount” or “a therapeutically effective amount” varies from subject to subject, due to variation in metabolism of the autologous tumor cell vaccine, age, weight, general condition of the subject, the condition being treated, the severity of the condition being treated, and the judgment of the prescribing physician.

As used herein, the terms “subject,” “individual,” and “patient” are used interchangeably. None of the terms are to be interpreted as requiring the supervision of a medical professional (e.g., a doctor, nurse, physician's assistant, orderly, hospice worker). As used herein, the subject is any animal, including mammals (e.g., a human or non-human animal) and non-mammals. In one embodiment of the methods and autologous tumor cell vaccines provided herein, the mammal is a human.

As used herein, the terms “treat,” “treating,” or “treatment,” and other grammatical equivalents, including, but not limited to, alleviating, abating, or ameliorating one or more symptoms of a disease or condition, ameliorating, preventing or reducing the appearance, severity, or frequency of one or more additional symptoms of a disease or condition, ameliorating or preventing the underlying metabolic causes of one or more symptoms of a disease or condition, inhibiting the disease or condition, such as, for example, arresting the development of the disease or condition, relieving the disease or condition, causing regression of the disease or condition, relieving a condition caused by the disease or condition, preventing relapse of the disease or condition, or inhibiting the symptoms of the disease or condition either prophylactically and/or therapeutically.

As used herein the term “nucleic acid” or “nucleic acid molecule” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. In some aspects, nucleic acid molecules are composed of monomers that are naturally-occurring nucleotides (such as DNA and RNA), or analogs of naturally-occurring nucleotides (e.g., α-enantiomeric forms of naturally-occurring nucleotides), or a combination of both. In some aspects, modified nucleotides have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, in some aspects, the entire sugar moiety is replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. In some aspects, nucleic acid monomers are linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. In some aspects, the term “nucleic acid” or “nucleic acid molecule” also includes so-called “peptide nucleic acids,” which comprise naturally-occurring or modified nucleic acid bases attached to a polyamide backbone. In some aspects, nucleic acids are single stranded or double stranded.

As used herein, the term “expression vector” refers to nucleic acid molecules encoding a gene that is expressed in a host cell. In some aspects, an expression vector comprises a transcription promoter, a gene, and a transcription terminator. In some aspects, gene expression is placed under the control of a promoter, and such a gene is said to be “operably linked to” the promoter. In some aspects, a regulatory element and a core promoter are operably linked if the regulatory element modulates the activity of the core promoter. As used herein, the term “promoter” refers to any DNA sequence which, when associated with a structural gene in a host yeast cell, increases, for that structural gene, one or more of 1) transcription, 2) translation or 3) mRNA stability, compared to transcription, translation or mRNA stability (longer half-life of mRNA) in the absence of the promoter sequence, under appropriate growth conditions.

As used herein the term “bi-functional” refers to a shRNA having two mechanistic pathways of action, that of the siRNA and that of the miRNA. The term “traditional” shRNA refers to a DNA transcription derived RNA acting by the siRNA mechanism of action. The term “doublet” shRNA refers to two shRNAs, each acting against the expression of two different genes but in the “traditional” siRNA mode.

As used herein the term “dry powder” refers to a fine particulate composition, with particles of mean mass diameter selected capable of being borne by a stream of air or gas, the dry powder not being suspended or dissolved in a propellant, carrier or other liquid. “Dry powder” does not necessarily imply the complete absence of water molecules from the formulation. In certain instances, the dry powder is a lyophilized particle or plurality of particles.

As used herein the term “aerosol” or “aerosolized” is meant to refer to dispersions in air of solid or liquid particles. In general, such particles have low settling velocities and relative airborne stability. In certain aspects, the particle size distribution is between 0.01 μm and 15 μm. By “agent” is meant any small molecule chemical compound, antibody, nucleic acid molecule, or polypeptide, or fragments thereof. A dry powder may be aerosolized using conventional dry powder inhalers.

### II. EMBODIMENTS

A. Inhalable Dosage Forms

The present disclosure provides inhalable dosage forms, comprising: a. an expression vector comprising i. a first insert comprising a nucleic acid sequence encoding a Granulocyte Macrophage Colony Stimulating Factor (GM-CSF) sequence; and ii. a second insert comprising a nucleic acid sequence encoding at least one bi-functional short hairpin RNA (bi-shRNA) capable of hybridizing to a region of a mRNA transcript encoding furin, to inhibit furin expression via RNA interference wherein the bi-shRNA incorporates cleavage dependent siRNA and cleavage independent miRNA motifs; and b. at least one stabilizing excipient.

Compositions and methods are disclosed for delivering dry powder formulations containing polynucleotides into a subject's respiratory tract including the lungs. The methods find use in the delivery of nucleic acid (e.g. DNA and RNA) expression vectors into airway epithelial cells, alveoli pulmonary macrophages and other cells in the respiratory tract (including the oropharynx nose nasopharynx). RNA polynucleotides may include shRNA, siRNA, miRNA and combinations thereof. These disclosed methods may be used for optimization of transfection efficiency and expression in vivo, treating lung cancer, reducing or eliminating tumors and for generating an immune response or inducing immunological tolerance.

In one aspect, the inhalable dosage particles are made using methods to produce stable micron and submicron particles comprising an expression vector. In certain aspects, the methods use a thin film freezing (TFF) technique (see, for example, U.S. Pat. No. 10,092,512). In TFF, liquid droplets typically fall from a given height and impact, spread, and freeze on a cooled solid substrate. In operation, a droplet falls from a given height, and impacts a spinning surface having a temperature of 223 K. As the droplet spreads out, a freezing front is formed in advance of the unfrozen liquid. TFF can be used to form high specific surface area powder of poorly water soluble drugs. TFF can be used for forming high surface area expression vector particles. TFF dry powder formulations can be delivered directly to the lungs via an inhaler.

Dry powder formulations typically comprise the expression vector in a dry, usually lyophilized, form with a particle size within a range for deposition within the alveolar region of the lung, typically having a diameter of from about 0.5 μm to about 15 μm or 0.5 μm to about 5 μm. Respirable powders containing an expression vector within the size range can be produced by a variety of conventional techniques, such as lyophilization, thin film freezing, jet-milling, spray-drying, solvent precipitation, and the like. Dry powders can then be administered to the patient or subject in conventional dry powder inhalers (DPI's) that use the patient's inspiratory breath through the device to disperse the powder or in air-assisted devices that use an external power source to disperse the powder into an aerosol cloud.

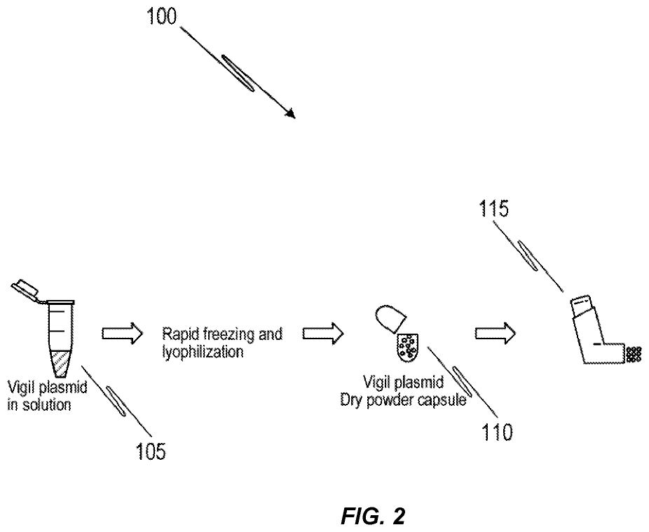

FIG. 2 shows a lyophilization method 100, wherein the expression vector is in solution 105, which can be rapidly frozen and lyophilized. After lyophilization, the lyophilized powder becomes a dry powder 110. A dry powder inhaler 115 can aerosolize the dry powder. Those skilled in the art will appreciate that lyophilization is a freeze-drying process in which water is sublimed from the composition after it is frozen. The particular advantage associated with the lyophilization process is that biologicals in an aqueous solution can be dried without elevated temperatures (thereby eliminating the adverse thermal effects), and then stored in a dry state where there are few stability problems. The lyophilized cake containing the expression vectors can be micronized using techniques known in the art to provide about 1 μm to about 10 μm, or 0.5 μm to about 5 μm sized particles.

Dry powder devices typically require a powder mass in the range from about 1 mg to 10 mg to produce a single aerosolized dose (“puff”). Since the required dose of the expression vector will generally be lower than this amount, the powder will typically be combined with a pharmaceutically acceptable dry bulking powder or stabilizing excipient. Dry bulking powders or stabilizing excipients include sucrose, lactose, trehalose, human serum albumin (HSA), and glycine. Other suitable dry bulking powders include cellobiose, dextrans, maltotriose, pectin, sodium citrate, sodium ascorbate, mannitol, and the like. Other stabilizing excipients include glucose, arabinose, maltose, saccharose, dextrose and/or a polyalcohol such as mannitol, maltitol, lactitol and sorbitol. In one embodiment, the sugar is trehalose. In some instances, suitable buffers and salts may be used to stabilize the expression vector in solution prior to particle formation. Suitable buffers include phosphate, citrate, acetate, and tris-HCl, typically at concentrations from about 5 mM to 50 mM. Suitable salts include sodium chloride, sodium carbonate, calcium chloride, and the like.

In one aspect, the expression vector is lyophilized with at least one stabilizing excipient prior to the administering, thereby producing lyophilized particles of about 0.5 μm to about 15 μm such as about 0.5 μm, 1 μm, 1.5 μm, 2 μm, 2.5 μm, 3 μm, 3.5 μm, 4 μm, 4.5 μm, 5 μm, 5.5 μm, 6 μm, 6.5 μm, 7 μm, 7.5 μm, 8 μm, 8.5 μm, 9 μm, 9.5 μm, 10 μm, 10.5 μm, 11 μm, 11.5 μm, 12 μm, 12.5 μm, 13 μm, 13.5 μm, 14 μm, 14.5 μm, and/or 15 μm. In some aspects, at least one stabilizing excipient is trehalose. In some aspects, the lyophilized particles are less than 5 μm in diameter. In some aspects, the lyophilized particles are from about 1 μm to about 3 μm in diameter. In some aspects, from about 1 mg to about 4 mg of the expression vector is administered to the individual. In some aspects, the administering comprises pulmonary delivery. In some aspects, the administering comprises pulmonary delivery of the expression vector to the individual or subject via a device selected from an inhaler or a nebulizer.

Dry powder aerosol compositions of the present disclosure can be used to transport polynucleotides via the lung into tumors, lymph, blood and macrophages or other cells of the body. In the methods of the present disclosure, delivery is generally achieved by controlling the size of the aerosolized particle containing an expression vector. In some aspects, methods are provided for delivering a dry powder aerosolized polynucleotide to the deep lung, i.e., the alveoli. In these aspects, a majority of the aerosolized, expression vector-containing particles have a size in the range of about 0.01 μm to about 10 μm such as 0.1 μm, 0.2 μm, 0.3 μm, 0.4 μm, 0.5 μm, 0.6 μm, 0.7 μm, 0.8 μm, 0.9 μm, 1 μm, 1.1 μm, 1.2 μm, 1.3 μm, 1.4 μm, 1.5 μm, 1.6 μm, 1.7 μm, 1.8 μm, 1.9 μm, 2 μm, 2.1 μm, 2.2 μm, 2.3 μm, 2.4 μm, 2.5 μm, 2.6 μm, 2.7 μm, 2.8 μm, 2.9 μm, 3 μm, 3.1 μm, 3.2 μm, 3.3 μm, 3.4 μm, 3.5 μm, 3.6 μm, 3.7 μm, 3.8 μm, 3.9 μm, 4 μm, 4.1 μm, 4.2 μm, 4.3 μm, 4.4 μm, 4.5 μm, 4.6 μm, 4.7 μm, 4.8 μm, 4.9 μm, 5 μm, 5.1 μm, 5.2 μm, 5.3 μm, 5.4 μm, 5.5 μm, 5.6 μm, 5.7 μm, 5.8 μm, 5.9 μm, 6 μm, 6.1 μm, 6.2 μm, 6.3 μm, 6.4 μm, 6.5 μm, 6.6 μm, 6.7 μm, 6.8 μm, 6.9 μm, 7 μm, 7.1 μm, 7.2 μm, 7.3 μm, 7.4 μm, 7.5 μm, 7.6 μm, 7.7 μm, 7.8 μm, 7.9 μm, 8 μm, 8.1 μm, 8.2 μm, 8.3 μm, 8.4 μm, 8.5 μm, 8.6 μm, 8.7 μm, 8.8 μm, 8.9 μm, 9 μm, 9.1 μm, 9.2 μm, 9.3 μm, 9.4 μm, 9.5 μm, 9.6 μm, 9.7 μm, 9.8 μm, 9.9 μm, and/or about 10 μm.

In some aspects, methods are provided for delivering an aerosolized dry powder expression vector polynucleotide to the central airways, i.e., the bronchi and bronchioles. In these aspects, a majority of the dry powder aerosol, polynucleotide-containing particles have a size in the range of about 4 μm to about 6 μm or about 5 μm. In still other aspects, methods are provided for delivering aerosolized particles to the upper respiratory tract, including the oropharyngeal region and the trachea. In certain aspects, the aerosol can be delivered to the alveoli if delivery to the circulatory system is desired. In this aspect, the particle size can be about 1 to about 5 microns, and can be a generally spherical shape.

Typically, the aerosol is created by forcing the drug formulation through a nozzle comprised of a porous membrane having pores in the range of about 0.25 to 6.0 microns in size. When the pores have this size the droplets that are formed will have a diameter about twice the diameter of the pore size. In order to ensure that the low resistance filter has the same or less flow resistance as the nozzle, the pore size and pore density of the filter should be adjusted as necessary with adjustments in pore size and pore density of the nozzle's porous membrane. Particle size can also be adjusted by adding heat to evaporate carrier. From the period of time from the formation of the aerosolized particles until the particles actually contact the lung surface, the size of the particles is subject to change due to increases or decrease in the amount of water in the formulation due to the relative humidity within the surrounding atmosphere.

In certain aspects, the term “carrier” means the material which forms the particle that contains the polynucleotide or plasmid being administered, along with other excipients, including bulk media, required for the safe and efficacious action of the polynucleotide. These carriers may be dissolved, dispersed or suspended in bulk media such as water, ethanol, saline solutions and mixtures thereof. Other bulk media can also be used provided that they can be formulated to create a suitable aerosol and do not adversely affect the active component or human lung tissue. Useful bulk media do not adversely interact with the polynucleotide and have properties which allow for the formation of aerosolized particles having a diameter in the range of 1.0 to 10 microns when a formulation comprising the bulk media.

For aqueous solutions, the polynucleotides may be dissolved in water or a buffer and formed into small particles to create an aerosol which is delivered to the subject. Alternatively, the polynucleotide may be in a solution or a suspension wherein a low-boiling point propellant is used as a carrier fluid. Suitable aerosol propellants include, but are not limited to, chlorofluorocarbons (CFC) and hydrofluorocarbons (HFC), a variety of which are known in the art. The polynucleotide may be in the form of a dry powder which is intermixed with an airflow in order to provide for delivery of polynucleotide to the subject. Respirable dry powders within the desired size range can be produced by a variety of conventional techniques, including jet-milling, spray-drying, solvent precipitation, and the like.

Dry powders are generally combined with a pharmaceutically acceptable dry bulking powder, with the polynucleotide or plasmid present usually at from about 1% to about 10% such as about 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, and/or 10% by weight. Examples of dry bulking powders include sucrose, lactose, trehalose, human serum albumin (HSA), and glycine. Other suitable dry bulking powders include cellobiose, dextrans, maltotriose, pectin, sodium citrate, sodium ascorbate, mannitol, and the like. Regardless of the formulation, it is preferable to create particles having a size in the desired range, which is related to airway diameter of the targeted region.

In certain instances, the dry powders for inhalation are formulated as pharmaceutically active substances with carrier particles of inert material such as lactose. The carrier particles are designed such that they have a larger diameter than the active substance particles making them easier to handle and store. The smaller active agent particles are bound to the surface of carrier particles during storage, but are torn from the carrier particles upon actuation of the device.

In some aspects, the polynucleotide and expression vector to be delivered can be formulated as a liposome or lipoplex formulation. Such complexes comprise a mixture of lipids which bind to genetic material (DNA or RNA) by means of cationic charge (electrostatic interaction). Cationic liposomes which may be used in the present invention include 3β-[N—(N′, N′-dimethyl-aminoethane)-carbamoyl]-cholesterol (DC-Chol), 1,2-bis(oleoyloxy-3-trimethylammonio-propane (DOTAP), lysinylphosphatidylethanolamine (L-PE), lipopolyamines such as lipospermine, N-(2-hydroxyethyl)-N,N-dimethyl-2,3-bis(dodecyloxy)-1-propanaminium bromide, dimethyl dioctadecyl ammonium bromide (DDAB), dioleoylphosphatidyl ethanolamine (DOPE), dioleoylphosphatidyl choline (DOPC), N(1,2,3-dioleyloxy) propyl-N,N,N-triethylammonium (DOTMA), DOSPA, DMRIE, GL-67, GL-89, Lipofectin, and Lipofectamine. Other suitable phospholipids which may be used include phosphatidylcholine, phosphatidylserine, phosphatidylethanolamine, sphingomyelin, phosphatidylinositol, and the like. Cholesterol may also be included.

B. Methods for Treating Tumors in the Lungs

In certain aspects, the present disclosure provides methods for treating lung cancer or a tumor in the lung of a subject. The method comprises administering to the subject an inhalable dosage form, comprising: an expression vector comprising (a) a first insert comprising a nucleic acid sequence encoding a Granulocyte Macrophage Colony Stimulating Factor (GM-CSF) sequence; and (b) a second insert comprising a nucleic acid sequence encoding at least one bi-functional short hairpin RNA (bi-shRNA) capable of hybridizing to a region of a mRNA transcript encoding furin, thereby inhibiting furin expression via RNA interference wherein the bi-shRNA incorporates cleavage dependent siRNA and cleavage independent miRNA motifs, and wherein the administering of the expression vector treats the cancer or tumor in the lung. In certain aspects, the second nucleic acid comprises a nucleic acid sequence of SEQ ID NO:2 or SEQ ID NO:9. In certain aspects, the expression vector is a bishRNA^{furin}/GMCSF expression vector. In certain aspects, the expression vector is a plasmid. The sequence of the plasmid is set forth as SEQ ID NO:3.

In some aspects, adjusting various parameters allows for specific location for particle delivery. These parameters include particle size, particle density, inspiratory flow rate, and total volume inhaled. Using the methods herein, it is possible to deliver the expression vector to the desired region(s) of the respiratory tract. For example, in some aspects, the aerosolized particles having a size in the range of about 1 μm to about 3 μm are delivered to the alveoli; aerosolized particles having a size in the range of about 4 μm to about 6 μm are delivered to the central airways; and aerosolized particles having a size in the range of about 7 μm to about 10 μm are delivered to the upper airways. Those of skill in the art know that these ranges are merely recommended and other ranges may also work.

In some aspects, the present disclosure provides a method for delivering an expression vector comprising a polynucleotide or plasmid to the respiratory tract of a subject. The method includes aerosolizing a dry powder formulation, to form a population of aerosolized particles, wherein the aerosolized particles have a diameter related to the diameter of airways in an area of the respiratory tract; and administering the aerosolized particles to the respiratory tract (i.e., inhaling the aerosolized particles into the respiratory tract) of the subject, wherein the diameter of the particles targets the particles to the region of the respiratory tract.

In certain aspects, the expression vector is a plasmid.

In certain aspects, the inhalable dosage form comprises particles comprising the expression vector and at least one stabilizing excipient selected from the group comprising glucose, arabinose, maltose, saccharose, dextrose, lactose, sucrose, trehalose, human serum albumin (HSA), and glycine.

In certain aspects, the at least one stabilizing excipient is trehalose.

In certain aspects, the particles are from about 0.5 μm to about 10 μm.

In certain aspects, wherein the particles are from about 1 μm to 3 μm in diameter.

C. Expression Vector

The inhalable composition comprises a. an expression vector comprising i. a first insert comprising a nucleic acid sequence encoding a Granulocyte Macrophage Colony Stimulating Factor (GM-CSF) sequence; and ii. a second insert comprising a nucleic acid sequence encoding at least one bi-functional short hairpin RNA (bi-shRNA) capable of hybridizing to a region of a mRNA transcript encoding furin, thereby inhibiting furin expression via RNA interference wherein the bi-shRNA incorporates cleavage dependent siRNA and cleavage independent miRNA motifs; and b. at least one stabilizing excipient.

In some embodiments, the GM-CSF in the expression vector is a human GM-CSF sequence. In some embodiments, the expression vector further comprises a promoter, e.g., the promoter is a cytomegalovirus (CMV) mammalian promoter. In some embodiments, the mammalian CMV promoter comprises a CMV immediate early (IE) 5′ UTR enhancer sequence and a CMV IE Intron A. In further embodiments, the expression vector further comprises a CMV enhancer sequence and a CMV intron sequence.

The first insert and the second insert in the expression vector can be operably linked to the promoter. In particular embodiments, the expression vector further comprises a nucleic acid sequence encoding a picornaviral 2A ribosomal skip peptide between the first and the second nucleic acid inserts.

In some embodiments, the expression vector comprises at least one bifunctional shRNA (bi-shRNA). In some embodiments, the bi-shRNA comprises a first stem-loop structure that comprises an siRNA component and a second stem-loop structure that comprises a miRNA component. In some embodiments, the bi-functional shRNA has two mechanistic pathways of action, that of the siRNA and that of the miRNA. Thus, in some embodiments, the bi-functional shRNA described herein is different from a traditional shRNA, i.e., a DNA transcription derived RNA acting by the siRNA mechanism of action or from a “doublet shRNA” that refers to two shRNAs, each acting against the expression of two different genes but in the traditional siRNA mode. In some embodiments, the bi-shRNA incorporates siRNA (cleavage dependent) and miRNA (cleavage-independent) motifs.

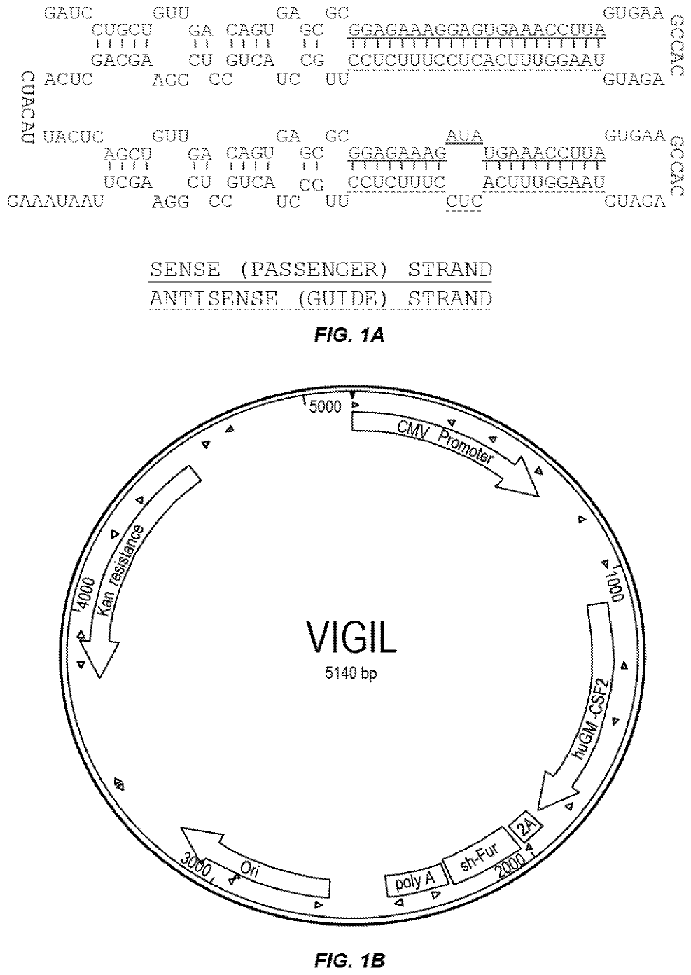

In some embodiments, the at least one bi-shRNA is capable of hybridizing to one of more regions of an mRNA transcript encoding furin. In some embodiments, the mRNA transcript encoding furin is a nucleic acid sequence of SEQ ID NO:1. In some embodiments, the one or more regions of the mRNA transcript encoding furin is selected from base sequences 300-318, 731-740, 1967-1991, 2425-2444, 2827-2851, and 2834-2852 of SEQ ID NO:1. In some embodiments, the expression vector targets the coding region of the furin mRNA transcript, the 3′ UTR region sequence of the furin mRNA transcript, or both the coding sequence and the 3′ UTR sequence of the furin mRNA transcript simultaneously. In some embodiments, the bi-shRNA comprises SEQ ID NO:2 or SEQ ID NO:9. In some embodiments, a bi-shRNA capable of hybridizing to one or more regions of an mRNA transcript encoding furin is referred to herein as bi-shRNA^{furin}. In some embodiments, the bi-shRNA^{furin} comprises or consists of two stem-loop structures with miR-30a backbone. In some embodiments, a first stem-loop structure of the two stem-loop structures comprises complementary guiding strand and passenger strand (FIG. 1A). In some embodiments, the second stem-loop structure of the two stem-loop structures comprises three mismatches in the passenger strand. In some embodiments, the three mismatches are at positions 9 to 11 in the passenger strand.

In certain aspects, the expression vector comprises:
a. a first insert comprising a nucleic acid sequence encoding a Granulocyte Macrophage Colony Stimulating Factor (GM-CSF) sequence; and
b. a second insert comprising two stem-loop structures each with a miR-30a loop; the first stem-loop structure has complete complementary guiding strand and passenger strand, while the second stem-loop structure has three basepair (bp) mismatches at positions 9 to 11 of the passenger strand.

In certain aspects, the guiding strand in the first stem-loop structure comprises the sequence of SEQ ID NO:6 and the passenger strand in the first stem-loop structure has the sequence of SEQ ID NO:5.

In certain aspects, the guiding strand in the second stem-loop structure comprises the sequence of SEQ ID NO:6 and the passenger strand in the second stem-loop structure has the sequence of SEQ ID NO:7.

In some embodiments, the expression vector plasmid can have a sequence that is at least 90% (e.g., 90%, 91%, 92%, 93%, 94%, 95%, 96%, 97%, 98%, 99%, or 100%) identical to the sequence of SEQ ID NO:3. The vector plasmid can comprise a first nucleic acid insert operably linked to a promoter, wherein the first insert encodes the GM-CSF cDNA, a second nucleic acid insert operably linked to the promoter, wherein the second insert encodes one or more short hairpin RNAs (shRNA) capable of hybridizing to a region of a mRNA transcript encoding furin, thereby inhibiting furin expression via RNA interference.

In SEQ ID NO:3, the bolded underlined portion is the GM-CSF sequence and the braided underlined in the furin shRNA portion of the sequence.

An expression vector comprising a first nucleic acid encoding GM-CSF and a second nucleic acid encoding at least one bifunctional short hairpin RNA (bi-shRNA) capable of hybridizing to a region of an mRNA transcript encoding furin is referred to as a bishRNA^{furin}/GMCSF expression vector.

In certain aspects, the second insert comprises a nucleic acid sequence of SEQ ID NO:2 or SEQ ID NO:9.

TABLE 1
Sequences

| SEQ ID | | |
|---|---|---|
| NO: | Description | Sequence |
| 1 | mRNA | GCGGGGAAGCAGCAGCGGCCAGGATGAATCCCAGGTG |
| | transcript | CTCTGGAGCTGGATGGTGAAGGTCGGCACTCTTCACCC |
| | encoding | TCCCGAGCCCTGCCCGTCTCGGCCCCATGCCCCCACCA |
| | furin | GTCAGCCCCGGGCCACAGGCAGTGAGCAGGCACCTGG |
| | | GAGCCGAGGCCCTGTGACCAGGCCAAGGAGACGGGCG |
| | | CTCCAGGGTCCCAGCCACCTGTCCCCCCCATGGAGCTG |
| | | AGGCCCTGGTTGCTATGGGTGGTAGCAGCAACAGGAA |
| | | CCTTGGTCCTGCTAGCAGCTGATGCTCAGGGCCAGAAG |
| | | GTCTTCACCAACACGTGGGCTGTGCGCATCCCTGGAGG |
| | | CCCAGCGGTGGCCAACAGTGTGGCACGGAAGCATGGG |
| | | TTCCTCAACCTGGGCCAGATCTTCGGGGACTATTACCA |
| | | CTTCTGGCATCGAGGAGTGACGAAGCGGTCCCTGTCGC |
| | | CTCACCGCCCGCGGCACAGCCGGCTGCAGAGGGAGCC |
| | | TCAAGTACAGTGGCTGGAACAGCAGGTGGCAAAGCGA |
| | | CGGACTAAACGGGACGTGTACCAGGAGCCCACAGACC |
| | | CCAAGTTTCCTCAGCAGTGGTACCTGTCTGGTGTCACTC |
| | | AGCGGGACCTGAATGTGAAGGCGGCCTGGGCGCAGGG |
| | | CTACACAGGGCACGGCATTGTGGTCTCCATTCTGGACG |
| | | ATGGCATCGAGAAGAACCACCCGGACTTGGCAGGCAA |
| | | TTATGATCCTGGGGCCAGTTTTGATGTCAATGACCAGG |
| | | ACCCTGACCCCCAGCCTCGGTACACACAGATGAATGAC |
| | | AACAGGCACGGCACACGGTGTGCGGGGGAAGTGGCTG |
| | | CGGTGGCCAACAACGGTGTCTGTGGTGTAGGTGTGGCC |
| | | TACAACGCCCGCATTGGAGGGGTGCGCATGCTGGATGG |
| | | CGAGGTGACAGATGCAGTGGAGGCACGCTCGCTGGGC |
| | | CTGAACCCCAACCACATCCACATCTACAGTGCCAGCTG |
| | | GGGCCCCGAGGATGACGGCAAGACAGTGGATGGGCCA |
| | | GCCCGCCTCGCCGAGGAGGCCTTCTTCCGTGGGGTTAG |
| | | CCAGGGCCGAGGGGGGCTGGGCTCCATCTTTGTCTGGG |
| | | CCTCGGGGAACGGGGGCCGGGAACATGACAGCTGCAA |
| | | CTGCGACGGCTACACCAACAGTATCTACACGCTGTCCA |
| | | TCAGCAGCGCCACGCAGTTTGGCAACGTGCCGTGGTAC |
| | | AGCGAGGCCTGCTCGTCCACACTGGCCACGACCTACAG |
| | | CAGTGGCAACCAGAATGAGAAGCAGATCGTGACGACT |
| | | GACTTGCGGCAGAAGTGCACGGAGTCTCACACGGGCA |
| | | CCTCAGCCTCTGCCCCCTTAGCAGCCGGCATCATTGCTC |
| | | TCACCCTGGAGGCCAATAAGAACCTCACATGGCGGGA |
| | | CATGCAACACCTGGTGGTACAGACCTCGAAGCCAGCCC |
| | | ACCTCAATGCCAACGACTGGGCCACCAATGGTGTGGGC |
| | | CGGAAAGTGAGCCACTCATATGGCTACGGGCTTTTGGA |
| | | CGCAGGCGCCATGGTGGCCCTGGCCCAGAATTGGACCA |
| | | CAGTGGCCCCCCAGCGGAAGTGCATCATCGACATCCTC |
| | | ACCGAGCCCAAAGACATCGGGAAACGGCTCGAGGTGC |
| | | GGAAGACCGTGACCGCGTGCCTGGGCGAGCCCAACCA |
| | | CATCACTCGGCTGGAGCACGCTCAGGCGCGGCTCACCC |
| | | TGTCCTATAATCGCCGTGGCGACCTGGCCATCCACCTG |
| | | GTCAGCCCCATGGGCACCCGCTCCACCCTGCTGGCAGC |
| | | CAGGCCACATGACTACTCCGCAGATGGGTTTAATGACT |
| | | GGGCCTTCATGACAACTCATTCCTGGGATGAGGATCCC |
| | | TCTGGCGAGTGGGTCCTAGAGATTGAAAACACCAGCG |
| | | AAGCCAACAACTATGGGACGCTGACCAAGTTCACCCTC |
| | | GTACTCTATGGCACCGCCCCTGAGGGGCTGCCCGTACC |
| | | TCCAGAAAGCAGTGGCTGCAAGACCCTCACGTCCAGTC |
| | | AGGCCTGTGTGGTGTGCGAGGAAGGCTTCTCCCTGCAC |
| | | CAGAAGAGCTGTGTCCAGCACTGCCCTCCAGGGTTCGC |
| | | CCCCCAAGTCCTCGATACGCACTATAGCACCGAGAATG |
| | | ACGTGGAGACCATCCGGGCCAGCGTCTGCGCCCCCTGC |
| | | CACGCCTCATGTGCCACATGCCAGGGGCCGGCCCTGAC |
| | | AGACTGCCTCAGCTGCCCCAGCCACGCCTCCTTGGACC |
| | | CTGTGGAGCAGACTTGCTCCCGGCAAAGCCAGAGCAG |
| | | CCGAGAGTCCCCGCCACAGCAGCAGCCACCTCGGCTGC |
| | | CCCCGGAGGTGGAGGCGGGGCAACGGCTGCGGGCAGG |
| | | GCTGCTGCCCTCACACCTGCCTGAGGTGGTGGCCGGCC |
| | | TCAGCTGCGCCTTCATCGTGCTGGTCTTCGTCACTGTCT |
| | | TCCTGGTCCTGCAGCTGCGCTCTGGCTTTAGTTTTCGGG |
| | | GGGTGAAGGTGTACACCATGGACCGTGGCCTCATCTCC |
| | | TACAAGGGGCTGCCCCCTGAAGCCTGGCAGGAGGAGT |
| | | GCCCGTCTGACTCAGAAGAGGACGAGGGCCGGGGCGA |
| | | GAGGACCGCCTTTATCAAAGACCAGAGCGCCCTCTGAT |
| | | GAGCCCACTGCCCACCCCCTCAAGCCAATCCCCTCCTT |
| | | GGGCACTTTTTAATTCACCAAAGTATTTTTTTATCTTGG |
| | | GACTGGGTTTGGACCCCAGCTGGGAGGCAAGAGGGGT |
| | | GGAGACTGCTTCCCATCCTACCCTCGGGCCCACCTGGC |
| | | CACCTGAGGTGGGCCCAGGACCAGCTGGGGCGTGGGG |
| | | AGGGCCGTACCCCACCCTCAGCACCCCTTCCATGTGGA |
| | | GAAAGGAGTGAAACCTTTAGGGCAGCTTGCCCCGGCCC |
| | | CGGCCCCAGCCAGAGTTCCTGCGGAGTGAAGAGGGGC |
| | | AGCCCTTGCTTGTTGGGATTCCTGACCCAGGCCGCAGC |
| | | TCTTGCCCTTCCCTGTCCCTCTAAAGCAATAATGGTCCC |
| | | ATCCAGGCAGTCGGGGGCTGGCCTAGGAGATATCTGA |
| | | GGGAGGAGGCCACCTCTCCAAGGGCTTCTGCACCCTCC |
| | | ACCCTGTCCCCCAGCTCTGGTGAGTCTTGGCGGCAGCA |
| | | GCCATCATAGGAAGGGACCAAGGCAAGGCAGGTGCCT |
| | | CCAGGTGTGCACGTGGCATGTGGCCTGTGGCCTGTGTC |
| | | CCATGACCCACCCCTGTGCTCCGTGCCTCCACCACCAC |
| | | TGGCCACCAGGCTGGCGCAGCCAAGGCCGAAGCTCTG |
| | | GCTGAACCCTGTGCTGGTGTCCTGACCACCCTCCCCTCT |
| | | CTTGCACCCGCCTCTCCCGTCAGGGCCCAAGTCCCTGTT |
| | | TTCTGAGCCCGGGCTGCCTGGGCTGTTGGCACTCACAG |
| | | ACCTGGAGCCCCTGGGTGGGTGGTGGGGAGGGGCGCT |
| | | GGCCCAGCCGGCCTCTCTGGCCTCCCACCCGATGCTGC |
| | | TTTCCCCTGTGGGGATCTCAGGGGCTGTTTGAGGATAT |
| | | ATTTTCACTTTGTGATTATTTCACTTTAGATGCTGATGA |
| | | TTTGTTTTTGTATTTTTAATGGGGGTAGCAGCTGGACTA |
| | | CCCACGTTCTCACACCCACCGTCCGCCCTGCTCCTCCCT |
| | | GGCTGCCCTGGCCCTGAGGTGTGGGGGCTGCAGCATGT |
| | | TGCTGAGGAGTGAGGAATAGTTGAGCCCCAAGTCCTGA |
| | | AGAGGCGGGCCAGCCAGGCGGGCTCAAGGAAAGGGGG |
| | | TCCCAGTGGGAGGGGCAGGCTGACATCTGTGTTTCAAG |
| | | TGGGGCTCGCCATGCCGGGGGTTCATAGGTCACTGGCT |
| | | CTCCAAGTGCCAGAGGTGGGCAGGTGGTGGCACTGAG |
| | | CCCCCCCAACACTGTGCCCTGGTGGAGAAAGCACTGAC |
| | | CTGTCATGCCCCCCTCAAACCTCCTCTTCTGACGTGCCT |
| | | TTTGCACCCCTCCCATTAGGACAATCAGTCCCCTCCCAT |
| | | CTGGGAGTCCCCTTTTCTTTTCTACCCTAGCCATTCCTG |
| | | GTACCCAGCCATCTGCCCAGGGGTGCCCCCTCCTCTCC |
| | | CATCCCCCTGCCCTCGTGGCCAGCCCGGCTGGTTTTGTA |
| | | AGATGCTGGGTTGGTGCACAGTGATTTTTTTCTTGTAAT |
| | | TTAAACAGGCCCAGCATTGCTGGTTCTATTTAATGGAC |
| | | ATGAGATAATGTTAGAGGTTTTAAAGTGATTAAACGTG |
| | | CAGACTATGCAAACCAG |
| 2 | bi-shRNA^{furin} | GAUCCUGCUGUUGACAGUGAGCGCGGAGAAAGGAGU |
| | | GAAACCUUAGUGAAGCCACAGAUGUAAGGUUUCACU |
| | | CCUUUCUCCUUGCCUACUGCCUCGGAAGCAGCUCACU |
| | | ACAUUACUCAGCUGUUGACAGUGAGCGCGGAGAAAG |
| | | AUAUGAAACCUUAGUGAAGCCACAGAUGUAAGGUUU |
| | | CACUCCUUUCUCCUUGCCUACUGCCUCGGAAGCUUAA |
| | | UAAAG |
| 3 | Plasmid | GGCCATTGCATACGTTGTATCCATATCATAATATGTAC |
| | | ATTTATATTGGCTCATGTCCAACATTACCGCCATGTTGA |
| | | CATTGATTATTGACTAGTTATTAATAGTAATCAATTACG |
| | | GGGTCATTAGTTCATAGCCCATATATGGAGTTCCGCGT |
| | | TACATAACTTACGGTAAATGGCCCGCCTGGCTGACCGC |
| | | CCAACGACCCCCGCCCATTGACGTCAATAATGACGTAT |
| | | GTTCCCATAGTAACGCCAATAGGGACTTTCCATTGACG |
| | | TCAATGGGTGGAGTATTTACGGTAAACTGCCCACTTGG |
| | | CAGTACATCAAGTGTATCATATGCCAAGTACGCCCCCT |
| | | ATTGACGTCAATGACGGTAAATGGCCCGCCTGGCATTA |
| | | TGCCCAGTACATGACCTTATGGGACTTTCCTACTTGGC |
| | | AGTACATCTACGTATTAGTCATCGCTATTACCATGGTG |
| | | ATGCGGTTTTGGCAGTACATCAATGGGCGTGGATAGCG |
| | | GTTTGACTCACGGGGATTTCCAAGTCTCCACCCCATTG |
| | | ACGTCAATGGGAGTTTGTTTTGGCACCAAAATCAACGG |
| | | GACTTTCCAAAATGTCGTAACAACTCCGCCCCATTGAC |
| | | GCAAATGGGCGGTAGGCGTGTACGGTGGGAGGTCTAT |
| | | ATAAGCAGAGCTCGTTTAGTGAACCGTCAGATCGCCTG |
| | | GAGACGCCATCCACGCTGTTTTGACCTCCATAGAAGAC |
| | | ACCGGGACCGATCCAGCCTCCGCGGCCGGGAACGGTG |
| | | CATTGGAACGCGGATTCCCCGTGCCAAGAGTGACGTAA |
| | | GTACCGCCTATAGACTCTATAGGCACACCCCTTTGGCT |
| | | CTTATGCATGCTATACTGTTTTTGGCTTGGGGCCTATAC |
| | | ACCCCCGCTTCCTTATGCTATAGGTGATGGTATAGCTTA |
| | | GCCTATAGGTGTGGGTTATTGACCATTATTGACCACTC |
| | | CAACGGTGGAGGGCAGTGTAGTCTGAGCAGTACTCGTT |
| | | GCTGCCGCGCGCGCCACCAGACATAATAGCTGACAGA |
| | | CTAACAGACTGTTCCTTTCCATGGGTCTTTTCTGCAGTC |
| | | ACCGTCGTCGACGGTATCGATAAGCTTGATATCGAATT |
| | | CCGCTGGAGGATGTGGCTGCAGAGCCTGCTGCTCTT |
| | | GGGCACTGTGGCCTGCAGCATCTCTGCACCCGCCC |
| | | GCTCGCCCAGCCCCAGCACGCAGCCCTGGGAGCAT |
| | | GTGAATGCCATCCAGGAGGCCCGGCGTCTCCTGAA |
| | | CCTGAGTAGAGACACTGCTGCTGAGATGAATGAAAC |
| | | AGTAGAAGTCATCTCAGAAATGTTTGACCTCCAGGA |
| | | GCCGACCTGCCTACAGACCCGCCTGGAGCTGTACA |
| | | AGCAGGGCCTGCGGGGCAGCCTCACCAAGCTCAAG |
| | | GGCCCCTTGACCATGATGGCCAGCCACTACAAGCA |
| | | GCACTGCCCTCCAACCCCGGAAACTTCCTGTGCAAC |
| | | CCAGACTATCACCTTTGAAAGTTTCAAAGAGAACCT |
| | | GAAGGACTTTCTGCTTGTCATCCCCTTTGACTGCTG |
| | | GGAGCCAGTCCAGGAGTGAGACCGGCCAGATGAGG |
| | | CTGGCCAAGCCGGGGAGCTGCTCTCTCATGAAACA |
| | | AGAGCTAGAAACTCAGGATGGTCATCTTGGAGGGA |
| | | CCAAGGGGTGGGCCACAGCCATGGTGGGAGTGGCC |
| | | TGGACCTGCCCTGGGCCACACTGACCCTGATACAG |
| | | GCATGGCAGAAGAATGGGAATATTTTATACTGACAG |
| | | AAATCAGTAATATTTATATATTTATATTTTTAAAATA |
| | | TTTATTTATTTATTTATTTAAGTTCATATTCCATATTT |
| | | ATTCAAGATGTTTTACCGTAATAATTATTATTAAAAA |
| | | TATGCTTCTAAAAAAAAAAAAAAAAAAAAAAACGGA |
| | | ATTCACGTGGGCCCGGTACCGTATACTCTAGAAGATCT |
| | | GGCAGCGGAGAGGGCAGAGGAAGTCTTCTAACATGCG |
| | | GTGACGTGGAGGAGAATCCCGGCCCTAGGATGTCTAG |
| | | AGCGGCCGCGGATCCTGCTGTTGACAGTGAGCGCGGA |
| | | GAAAGGAGTGAAACCTTAGTGAAGCCACAGATGTAAG |
| | | GTTTCACTCCTTTCTCCTTGCCTACTGCCTCGGAAGCAG |
| | | CTCACTACATTACTCAGCTGTTGACAGTGAGCGCGGAG |
| | | AAAGATATGAAACCTTAGTGAAGCCACAGATGTAAGG |
| | | TTTCACTCCTTTCTCCTTGCCTACTGCCTCGGAAGCTTA |
| | | ATAAAGGATCTTTTATTTTCATTGGATCCAGATCTTTTT |
| | | CCCTCTGCCAAAAATTATGGGGACATCATGAAGCCCCT |
| | | TGAGCATCTGACTTCTGGCTAATAAAGGAAATTTATTT |
| | | TCATTGCAATAGTGTGTTGGAATTTTTTGTGTCTCTCAC |
| | | TCGGAAGGACATATGGGAGGGCAAATCATTTAAAACA |
| | | TCAGAATGAGTATTTGGTTTAGAGTTTGGCAACATATG |
| | | CCCATTCTTCCGCTTCCTCGCTCACTGACTCGCTGCGCT |
| | | CGGTCGTTCGGCTGCGGCGAGCGGTATCAGCTCACTCA |
| | | AAGGCGGTAATACGGTTATCCACAGAATCAGGGGATA |
| | | ACGCAGGAAAGAACATGTGAGCAAAAGGCCAGCAAAA |
| | | GGCCAGGAACCGTAAAAAGGCCGCGTTGCTGGCGTTTT |
| | | TCCATAGGCTCCGCCCCCCTGACGAGCATCACAAAAAT |
| | | CGACGCTCAAGTCAGAGGTGGCGAAACCCGACAGGAC |
| | | TATAAAGATACCAGGCGTTTCCCCCTGGAAGCTCCCTC |
| | | GTGCGCTCTCCTGTTCCGACCCTGCCGCTTACCGGATAC |
| | | CTGTCCGCCTTTCTCCCTTCGGGAAGCGTGGCGCTTTCT |
| | | CATAGCTCACGCTGTAGGTATCTCAGTTCGGTGTAGGT |
| | | CGTTCGCTCCAAGCTGGGCTGTGTGCACGAACCCCCCG |
| | | TTCAGCCCGACCGCTGCGCCTTATCCGGTAACTATCGT |
| | | CTTGAGTCCAACCCGGTAAGACACGACTTATCGCCACT |
| | | GGCAGCAGCCACTGGTAACAGGATTAGCAGAGCGAGG |
| | | TATGTAGGCGGTGCTACAGAGTTCTTGAAGTGGTGGCC |
| | | TAACTACGGCTACACTAGAAGAACAGTATTTGGTATCT |
| | | GCGCTCTGCTGAAGCCAGTTACCTTCGGAAAAAGAGTT |
| | | GGTAGCTCTTGATCCGGCAAACAAACCACCGCTGGTAG |
| | | CGGTGGTTTTTTTGTTTGCAAGCAGCAGATTACGCGCA |
| | | GAAAAAAAGGATCTCAAGAAGATCCTTTGATCTTTTCT |
| | | ACGGGGTCTGACGCTCAGTGGAACGAAAACTCACGTTA |
| | | AGGGATTTTGGTCATGAGATTATCAAAAAGGATCTTCA |
| | | CCTAGATCCTTTTAAATTAAAAATGAAGTTTTAAATCA |
| | | ATCTAAAGTATATATGAGTAAACTTGGTCTGACAGTTA |
| | | CCAATGCTTAATCAGTGAGGCACCTATCTCAGCGATCT |
| | | GTCTATTTCGTTCATCCATAGTTGCCTGACTCGGGGGG |
| | | GGGGGGCGCTGAGGTCTGCCTCGTGAAGAAGGTGTTGC |
| | | TGACTCATACCAGGCCTGAATCGCCCCATCATCCAGCC |
| | | AGAAAGTGAGGGAGCCACGGTTGATGAGAGCTTTGTT |
| | | GTAGGTGGACCAGTTGGTGATTTTGAACTTTTGCTTTGC |
| | | CACGGAACGGTCTGCGTTGTCGGGAAGATGCGTGATCT |
| | | GATCCTTCAACTCAGCAAAAGTTCGATTTATTCAACAA |
| | | AGCCGCCGTCCCGTCAAGTCAGCGTAATGCTCTGCCAG |
| | | TGTTACAACCAATTAACCAATTCTGATTAGAAAAACTC |
| | | ATCGAGCATCAAATGAAACTGCAATTTATTCATATCAG |
| | | GATTATCAATACCATATTTTTGAAAAAGCCGTTTCTGTA |
| | | ATGAAGGAGAAAACTCACCGAGGCAGTTCCATAGGAT |
| | | GGCAAGATCCTGGTATCGGTCTGCGATTCCGACTCGTC |
| | | CAACATCAATACAACCTATTAATTTCCCCTCGTCAAAA |
| | | ATAAGGTTATCAAGTGAGAAATCACCATGAGTGACGA |
| | | CTGAATCCGGTGAGAATGGCAAAAGCTTATGCATTTCT |
| | | TTCCAGACTTGTTCAACAGGCCAGCCATTACGCTCGTC |
| | | ATCAAAATCACTCGCATCAACCAAACCGTTATTCATTC |
| | | GTGATTGCGCCTGAGCGAGACGAAATACGCGATCGCTG |
| | | TTAAAAGGACAATTACAAACAGGAATCGAATGCAACC |
| | | GGCGCAGGAACACTGCCAGCGCATCAACAATATTTTCA |
| | | CCTGAATCAGGATATTCTTCTAATACCTGGAATGCTGTT |
| | | TTCCCGGGGATCGCAGTGGTGAGTAACCATGCATCATC |
| | | AGGAGTACGGATAAAATGCTTGATGGTCGGAAGAGGC |
| | | ATAAATTCCGTCAGCCAGTTTAGTCTGACCATCTCATCT |
| | | GTAACATCATTGGCAACGCTACCTTTGCCATGTTTCAG |
| | | AAACAACTCTGGCGCATCGGGCTTCCCATACAATCGAT |
| | | AGATTGTCGCACCTGATTGCCCGACATTATCGCGAGCC |
| | | CATTTATACCCATATAAATCAGCATCCATGTTGGAATTT |
| | | AATCGCGGCCTCGAGCAAGACGTTTCCCGTTGAATATG |
| | | GCTCATAACACCCCTTGTATTACTGTTTATGTAAGCAG |
| | | ACAGTTTTATTGTTCATGATGATATATTTTTATCTTGTG |
| | | CAATGTAACATCAGAGATTTTGAGACACAACGTGGCTT |
| | | TCCCCCCCCCCCCATTATTGAAGCATTTATCAGGGTTAT |
| | | TGTCTCATGAGCGGATACATATTTGAATGTATTTAGAA |
| | | AAATAAACAAATAGGGGTTCCGCGCACATTTCCCCGAA |
| | | AAGTGCCACCTGACGTCTAAGAAACCATTATTATCATG |
| | | ACATTAACCTATAAAAATAGGCGTATCACGAGGCCCTT |
| | | TCGTCTCGCGCGTTTCGGTGATGACGGTGAAAACCTCT |
| | | GACACATGCAGCTCCCGGAGACGGTCACAGCTTGTCTG |
| | | TAAGCGGATGCCGGGAGCAGACAAGCCCGTCAGGGCG |
| | | CGTCAGCGGGTGTTGGCGGGTGTCGGGGCTGGCTTAAC |
| | | TATGCGGCATCAGAGCAGATTGTACTGAGAGTGCACCA |
| | | TATGCGGTGTGAAATACCGCACAGATGCGTAAGGAGA |
| | | AAATACCGCATCAGATTGGCTATT |
| 4 | HUMAN | AGTACACAGAGAGAAAGGCTAAAGTTCTCTGGAGGAT |
| | GM-CSF | GTGGCTGCAGAGCCTGCTGCTCTTGGGCACTGTGGCCT |
| | SEQUENCE | GCAGCATCTCTGCACCCGCCCGCTCGCCCAGCCCCAGC |
| | | ACGCAGCCCTGGGAGCATGTGAATGCCATCCAGGAGG |
| | | CCCGGCGTCTCCTGAACCTGAGTAGAGACACTGCTGCT |
| | | GAGATGAATGAAACAGTAGAAGTCATCTCAGAAATGT |
| | | TTGACCTCCAGGAGCCGACCTGCCTACAGACCCGCCTG |
| | | GAGCTGTACAAGCAGGGCCTGCGGGGCAGCCTCACCA |
| | | AGCTCAAGGGCCCCTTGACCATGATGGCCAGCCACTAC |
| | | AAGCAGCACTGCCCTCCAACCCCGGAAACTTCCTGTGC |
| | | AACCCAGATTATCACCTTTGAAAGTTTCAAAGAGAACC |
| | | TGAAGGACTTTCTGCTTGTCATCCCCTTTGACTGCTGGG |
| | | AGCCAGTCCAGGAGTGAGACCGGCCAGATGAGGCTGG |
| | | CCAAGCCGGGGAGCTGCTCTCTCATGAAACAAGAGCTA |
| | | GAAACTCAGGATGGTCATCTTGGAGGGACCAAGGGGT |
| | | GGGCCACAGCCATGGTGGGAGTGGCCTGGACCTGCCCT |
| | | GGGCCACACTGACCCTGATACAGGCATGGCAGAAGAA |
| | | TGGGAATATTTTATACTGACAGAAATCAGTAATATTTA |
| | | TATATTTATATTTTTAAAATATTTATTTATTTATTTATTT |
| | | AAGTTCATATTCCATATTTATTCAAGATGTTTTACCGTA |
| | | ATAATTATTATTAAAAATATGCTTCTACTTG |
| 5 | MIR-30A | GUGAAGCCACAGAUG |
| | LOOP | |
| 6 | GUIDE | UAAGGUUUCACUCCUUUCUCC |
| | STRAND IN | |
| | BOTH FIRST | |
| | AND | |
| | SECOND | |
| | STEM-LOOP | |
| | STRUCTURE | |
| | S IN BI- | |
| | SHRNA^{FURIN} | |
| 7 | PASSENGER | GGAGAAAGGAGUGAAACCUUA |
| | STRAND IN | |
| | FIRST STEM- | |
| | LOOP | |
| | STRUCTURE | |
| | IN BI- | |
| | SHRNA^{FURIN} | |
| 8 | PASSENGER | GGAGAAAGAUAUGAAACCUUA |
| | STRAND IN | |
| | SECOND | |
| | STEM-LOOP | |
| | STRUCTURE | |
| | IN BI- | |
| | SHRNA^{FURIN} | |
| 9 | bi-shRNA^{furin} | GAUCCUGCUGUUGACAGUGAGCGCGGAGAAAGGAGU |
| | | GAAACCUUAGUGAAGCCACAGAUGUAAGGUUUCACU |
| | | CCUUUCUCCUUGCCUACUGCCUCGGAAGCAGCUCACU |
| | | ACAUUACUCAGCUGUUGACAGUGAGCGCGGAGAAAG |
| | | AUAUGAAACCUUAGUGAAGCCACAGAUGUAAGGUUU |
| | | CACUCCUUUCUCCUUGCCUACUGCCUCGGAAGCUUAA |
| | | UAAAGGAUCUUUUAUUUUCAUUGGAUC |

In certain aspects, the at least one stabilizing excipient is trehalose. In some aspects, the expression vector is a plasmid.

In certain aspects, the GM-CSF is a human GM-CSF sequence.

In certain aspects, the expression vector further comprises a promoter.

In certain aspects, the promoter is a cytomegalovirus (CMV) mammalian promoter.

In certain aspects, the expression vector further comprises a CMV enhancer sequence and a CMV intron sequence.

In certain aspects, the first insert and the second insert are operably linked to the promoter.

In certain aspects, the expression vector further comprises a nucleic acid sequence encoding a picornaviral 2A ribosomal skip peptide between the first and the second nucleic acid inserts.

In some aspects, the GM-CSF is human GM-CSF In certain instances, the first nucleic acid encoding GM-CSF is rHGM-CSF (recombinant human granulocyte-macrophage colony stimulating factor) cDNA. The accession number for Homo sapiens colony stimulating factor 2 (CSF2). mRNA is NM_000758 and is SEQ ID NO:4 A reverse transcriptase is used to synthesize double-stranded DNA, which is a complimentary copy of the mRNA i.e., the complimentary sequence to SEQ ID NO:4. In some aspects, a nucleotide sequence encoding a picornaviral 2A ribosomal skip peptide sequence is intercalated between the first and the second nucleic acid inserts.

Granulocyte-macrophage colony-stimulating factor, often abbreviated to GM-CSF, is a protein secreted by macrophages, T cells, mast cells, endothelial cells and fibroblasts. When integrated as a cytokine transgene, GM-CSF enhances presentation of cancer vaccine peptides, tumor cell lysates, or whole tumor cells from either autologous or established allogeneic tumor cell lines. GM-CSF induces the differentiation of hematopoietic precursors and attracts them to the site of vaccination. GM-CSF also functions as an adjuvant for dendritic cell maturation and activational processes. However. GM-CSF-mediated immunosensitization can be suppressed by tumor produced and/or secreted different isoforms of transforming growth factor beta (TGF-β). The TGF-β family of multifunctional proteins possesses well known immunosuppressive activities. The three known TGF-β ligands (TGF-β1, β2, and β3) are ubiquitous in human cancers. TGF-β overexpression correlates with tumor progression and poor prognosis. Elevated TGF-β levels within the tumor microenvironment are linked to an anergic antitumor response. TGF-β inhibits GM-CSF induced maturation of dendritic cells and their expression of MI-IC class II and co-stimulatory molecules. This negative impact of TGF-β on GM-CSF-mediated immune activation supports the rationale of depleting TGF-β secretion in GM-CSF-based cancer cell vaccines.

All mature isoforms of TGF-β require furin-mediated limited proteolytic cleavage for proper activity Furin, a calcium-dependent serine endoprotease, is a member of the subtilisin-like proprotein convertase family Furin is best known for the functional activation of TGF-β with corresponding immunoregulatory ramifications.

High levels of furin have been demonstrated in virtually all cancer lines. Up to a 10-fold higher level of TGF-β1 may be produced by human colorectal, lung cancer, and melanoma cells, and likely impact the immune tolerance state by a higher magnitude. The presence of furin in tumor cells likely contributes significantly to the maintenance of tumor directed TGF-β peripheral immune tolerance. Hence furin knockdown (via RNA interference mechanism) represents an attractive approach for optimizing GM-CSF-mediated immunosensitization.

In some aspects, the furin-knockdown bi-functional RNA is described in U.S. Pat. No. 9,157,084, which is incorporated herein by reference. Compositions and methods to attenuate the immunosuppressive activity of TGF-β through the use of bi-functional shRNAs is described. The bi-functional shRNAs knocks down the expression of furin in cancer cells to augment tumorc antigen expression, presentation, and processing through expression of the GM-CSF transgene.

As such, U.S. Pat. No. 9,157,084 discloses: an expression vector comprising: a first nucleic acid insert operably linked to a promoter, wherein the first insert encodes a human Granulocyte Macrophage Colony Stimulating Factor (GM-CSF) cDNA; and a second nucleic acid insert operably linked to the promoter, wherein the second insert encodes one or more bifunctional short hairpin RNAs (shRNA) capable of hybridizing to one of more regions of a mRNA transcript encoding furin, wherein at least one of the regions is selected from base sequences 300-318, 731-740, 1967-1991, 2425-2444, 2827-2851 or 2834-2852 of SEQ ID NO:1, thereby inhibiting furin expression via RNA interference, wherein each bifunctional short hairpin RNA comprises a first stem-loop structure that comprises an siRNA component and a second stem-loop structure that comprises a miRNA component and wherein the shRNA incorporates siRNA (cleavage dependent) and miRNA (cleavage-independent) motifs.

Advantageously, the expression vector works by using a combined approach of depleting multiple immunosuppressive TGF-β isoforms by furin knockdown, in order to maximize the immune enhancing effects of the incorporated GM-CSF transgene on autologous tumor antigen sensitization.

In certain aspects, the expression vector described herein can be used as vaccine. In this regard, the vaccine is described in U.S. Pat. No. 9,132,146, which is incorporated herein by reference. The patent describes compositions and methods for cancer treatment. The autologous cancer vaccine is genetically modified for Furin knockdown and GM-CSF expression. The vaccine attenuates the immunosuppressive activity of TGF-β through the use of bi-functional shRNAs to knock down the expression of furin in cancer cells, and augments tumor antigen expression, presentation, and processing through expression of the GM-CSF transgene.

As such, U.S. Pat. No. 9,132,146 discloses: An autologous cell vaccine comprising: a bishRNA^{furin}/GMCSF expression vector plasmid, wherein the vector plasmid comprises a first nucleic acid insert operably linked to a promoter, wherein the first insert encodes a Granulocyte Macrophage Colony Stimulating Factor (GM-CSF) cDNA; a second nucleic acid insert operably linked to the promoter, wherein the second insert encodes one or more short hairpin RNAs (shRNA) capable of hybridizing to a region of a mRNA transcript encoding furin, thereby inhibiting furin expression via RNA interference wherein the shRNA incorporates cleavage dependent siRNA and cleavage independent miRNA motifs.

In certain aspects, the expression vector described herein is also disclosed in U.S. Pat. No. 9,790,518, which is incorporated herein by reference. Compositions and methods for cancer treatment are disclosed, using an autologous cancer vaccine genetically modified for Furin knockdown and GM-CSF expression. The vaccine described attenuates the immunosuppressive activity of TGF-β through the use of bi-functional shRNAs to knock down the expression of furin in cancer cells, and to segment tumor antigen expression, presentation, and processing through expression of the GM-CSF transgene.

As such, U.S. Pat. No. 9,790,518 discloses: A method of treating a cancer in an individual in need thereof comprising: a. transfecting an autologous tumor cell from the individual with an expression vector comprising: i. a first insert comprising a nucleic acid sequence encoding a Granulocyte Macrophage Colony Stimulating Factor (GM-CSF) sequence; and ii. wherein the second insert encodes one or more short hairpin RNAs (shRNA) capable of hybridizing to a region of a mRNA transcript encoding furin; and b. administering the transfected tumor cell to the individual.

In certain aspects, the expression vector is disclosed in U.S. Pat. No. 9,695,422, which discloses compositions and methods to attenuate the immunosuppressive activity of TGF-β through the use of bi-functional shRNAs is described herein. The bi-functional shRNAs of the present invention knocks down the expression of furin in cancer cells to augment tumor antigen expression, presentation, and processing through expression of the GM-CSF transgene.

As such, U.S. Pat. No. 9,695,422 discloses: An expression vector comprising: a first nucleic acid insert operably linked to a promoter, wherein the first insert encodes a Granulocyte Macrophage Colony Stimulating Factor (GM-CSF) cDNA sequence; and a second nucleic acid insert operably linked to the promoter, wherein the second insert encodes a bi-functional short hairpin RNA (bi-shRNA), wherein the bi-shRNA comprises: (a) a first stem loop structure comprising (i) a first guide sequence capable of hybridizing to a region of a mRNA transcript corresponding to base sequences 300-318, 731-740, 1967-1991, 2425-2444, 2827-2851, or 2834-2852 of SEQ ID NO:1; and (ii) a first passenger sequence fully complementary to the first guide sequence; and (b) a second stem loop structure comprising (i) a second guide sequence capable of hybridizing to a region of an mRNA transcript corresponding to base sequences 300-318, 731-740, 1967-1991, 2425-2444, 2827-2851, or 2834-2852 of SEQ ID NO:1; and (ii) a second passenger sequence partially complementary to the second guide sequence.

In certain aspects, a subject will be administered a dosing regimen of 1-3 doses at a frequency of 2-3 weeks apart. Dose will be calculated based on average lung weight and concentration of plasmid used in oncology studies which has shown efficacy and safety, which for a human will be an average lung with of 1 kg and a dose of 4 mg.

In certain aspects, the compositions disclosed herein are used in combination with nivolumab (Opdivo) or in combination with nivolumab together in combination with ipilimumab (Yervoy) for the first-line treatment of patients with metastatic or recurrent non-small cell lung cancer (NSCLC) with no EGFR or ALK genomic tumor aberrations. In certain aspects, the compositions herein can be used with nivolumab (Opdivo), ipilimumab (Yervoy), and two cycles of platinum-doublet chemotherapy as frontline treatment for patients with metastatic or recurrent non-small cell lung cancer (NSCLC) who have no EGFR or ALK genomic tumor aberrations.

Unit dosage forms may be within, for example, ampules and vials, which may include a liquid composition, or a composition in a freeze-dried or lyophilized state: a sterile liquid carrier, for example, can be added prior to administration or delivery in vivo. Individual unit dosage forms can be included in multi-dose kits or containers. Pharmaceutical formulations can be packaged in single or multiple unit dosage form for ease of administration and uniformity of dosage.

### III. EXAMPLES

Example 1 illustrates the lyophilization procedure to produce Lyophilized Vigil® plasmid particles.

Anti-furin therapeutic: GM-CSF bi-shRNAa^{furin} plasmid (VP) SEQ ID NO:3.

VP constructed by Gradalis, Inc. (TX, USA), consists of two stem-loop structures with a miR-30a backbone. The bi-shRNA^{furin} DNA as shown in FIG. 1A uses a single targeted site to induce both mRNA cleavage and sequestration in P-bodies (translational silencing) and/or GW-bodies (repositories). By the use of this proprietary process, the encoding bi-shRNA can accommodate mature shRNA loaded onto more than one type of RNA induced silencing complex (RISC). Also, with the bi-shRNA^{furin} molecular design focusing on a single site potential toxic effects are reduced. Targeting of multiple sites increases chance for a ‘seed sequence’ being induced and leading to off-target effect that could result in increased clinical toxicity. Synthetic complementing and interconnecting oligonucleotides via DNA ligation were used to assemble the two stem-loop double stranded DNA sequences. The 241 base pair DNA constructed with Bam HI sites at both ends was inserted into the Bam HI site of a prior clinically validated plasmid called TAG in which was removed a TGFβ2 antisense DNA sequence and placed the bi shRNA^{furin}-GMCSF DNA sequence. Orientation of the inserted DNA was validated by the appropriate PCR primer pairs designed to screen for the shRNA insert and orientation. Safety profile defined with the prior TAG clinical therapeutic was used to support clinical advancement of VP in experimental cancer management testing under FDA guidance. Vigil is designed with the mammalian promoter cytomegalovirus [CMV] that drives the cassette.

In between the GM-CSF gene (with a stop codon) and furin bi-shRNA there is a 2A ribosomal skip peptide followed by a rabbit poly-A tail. The picornaviral 2A sequence allows the production of two proteins from one open reading frame, by causing ribosomes to skip formation of a peptide bond at the junction of the 2A and downstream sequences. Since the 2A linker has previously been demonstrated to be effective for generating similar expression levels of GM-CSF and anti-TGFβ transcripts with the TAG vaccine and robust activity in product release testing of therapeutic effector components expressed with this plasmid design along with clinical benefit and safety has been observed, the same design for VP was maintained. Transient expression of bi shRNA^{furin} GM-CSF plasmid and diluted expressive cell numbers in patients would not be expected to approach continuous toxic effect of transgenic models.

Gradalis has been clinically testing this plasmid since 2009. Aldevron (ND, USA) and Waisman (WI, USA) have participated in lot manufacturing. This plasmid, which consists of a bi shRNA^{furin} DNA sequence and a GM-CSF DNA sequence (FIG. 1B; SEQ ID NO:3), has been validated for FDA registration trial based on significant cancer patient benefit for use of transfection (via electroporation) into an autologous tumor for vaccine (Vigil) construction in Ewing's sarcoma. Additional benefit has also been suggested in a variety of other cancer types in Phase I and II testing, most notably ovarian cancer. The current active lot of Vigil plasmid was manufactured in 2015 by Waisman. The plasmid concentration per vial is 2.2 mg/ml which provides 770 μg of plasmid. Yearly stability testing has passed all measures of evaluation under US FDA review. Recent double blind randomized control trial involving 25 nationally acclaimed sites unblinded and revealed OS advantage (HR: 0.417, p=0.020) and RFS advantage (HR: 0.459, p=0.007; stratified Cox's proportional Hazard Model).

FIG. 2 shows a lyophilization method 100, wherein the expression vector is in solution 105, which can be rapidly frozen and lyophilized. After lyophilization, the lyophilized powder comprising Vigil becomes a dry powder 110. A dry powder inhaler 115 can aerosolize the dry powder. Those skilled in the art will appreciate that lyophilization is a freeze-drying process in which water is sublimed from the composition after it is frozen. The particular advantage associated with a lyophilization process is that biologicals in an aqueous solution can be dried without elevated temperatures (thereby eliminating the adverse thermal effects), and then stored in a dry state where there are few stability problems. The lyophilized cake containing the expression vectors can be micronized using techniques known in the art to provide 1 μm to 10 μm sized particles.

Example 2 illustrates the transfection for Lyophilized Vigil® Particles in two cell lines.

The transfection efficiencies of the Lyophilized Vigil® Particles was investigated in two cell lines CCL-247 and RD-ES. The results are in the Table below.

| Time Point | Day 2 | | | Day 3 | | |
|---|---|---|---|---|---|---|
| Treatment | Zap + | Lipo + | Lipo + Lyo | Zap + Frozen | Lipo + | Lipo + Lyo |
| Condition | Frozen DNA | Frozen DNA | DNA (5% Tre) | DNA | Frozen DNA | DNA (5% Tre) |
| GFP | 65% | 50% | 70% | 69% | 50% | 52% |
| (Transfection | | | | | | |
| efficiency) | | | | | | |
| GM-CSF | 1.85 e6 | 3.01 e6 | 3.82 e6 | 3.95 e6 | 4.84 e6 | 5.09 e6 |
| pg/mL | | | | | | |
| TGF-81 | 54% | 23% | 35% | 59% | 30% | 41% |
| (Knock | | | | | | |
| Down) | | | | | | |

Several Lipo 3000 + Lyo DNA conditions tested
Lyo DNA + 5% Trehalose yielded best results from Lyo groups
Transfection efficiency for all test conditions ranged 50-70%
Electroporation (280 V) with Frozen DNA yielded best TGF-B1 Knock Down (apx 1.4-1.5 more: Zap vs Lyo 5% Tre)
Lipo 3000 + Lyo DNA with 5% Trehalose yielded best GM-CSF expression (apx 1.2-2.0 more: Lyo 5% Tre cs Zap)

CCL-247 is a human colorectal carcinoma cell line initiated from an adult male. RD-ES is a human bone Ewing's sarcoma cell line. Admix 100 μL DNA plasmid and 100 μL of a 10% Sucrose solution to generate 200 μL solution of 5% Sucrose and the DNA plasmid in a vial. Seal the vial and freeze at −80° C. Lyophilize the sample at −23° C. The DNA sample can be checked for quality using a restriction enzyme digest and Agarose Gel Electrophoresis (AGE).

Cells can be transfected using electroporation. Electroporation, which uses pulsed electrical fields, can be used to introduce DNA into a variety of animal cells. Here, the cells are electroporated at 280 V, 1000 μF.

After transfection, the transfected cells are then cultured in 6-well plates. 100 μL of the cells are transfer to a 6-well plate and 900 μL X-Vivo media is added to achieve 1 M cells in 1 mL. In addition, 100 μL of cells is transferred to a clean 6-well plate and add 900 μL FBS media to achieve 1 M cells in 1 mL. The 6-well plate is incubated at 37° C. for 3 days. The supernatant is immunoassayed for furin knock down and expression of GM-CSFs.

Various conditions were tested. The Lipo 3000 and “Lyo DNA” with 5% trehalose yielded the best GM-CSF expression.

Example 3 illustrates the transfection for thin film freezing (TFF) Vigil® Particles in various cell lines.

In this example, the TFF Vigil particles were placed in Cell Culture and tested in CCL-247, RD-ES, and Calu-3 cells. In addition to the cell lines used above, Calu-3 cells were also used. Calu-3 is a human lung cancer cell line, which are epithelial and can act as respiratory models in preclinical applications. Calu-3 cells are commonly used as both in vitro and in vivo models for drug development against lung cancer. The cells have been used in studies of pulmonary drug delivery, demonstrating a capacity to intake low molecular weight substances. Calu-3 cells have served as respiratory models for air intake and lung injury due to their responsiveness to foreign substances.

The thin film freezing (TFF) Vigil particles used were 5% powder or a 70/30 mannitol/leucine powder. The nucleic acid was checked before use by agarose gel electrophoresis.

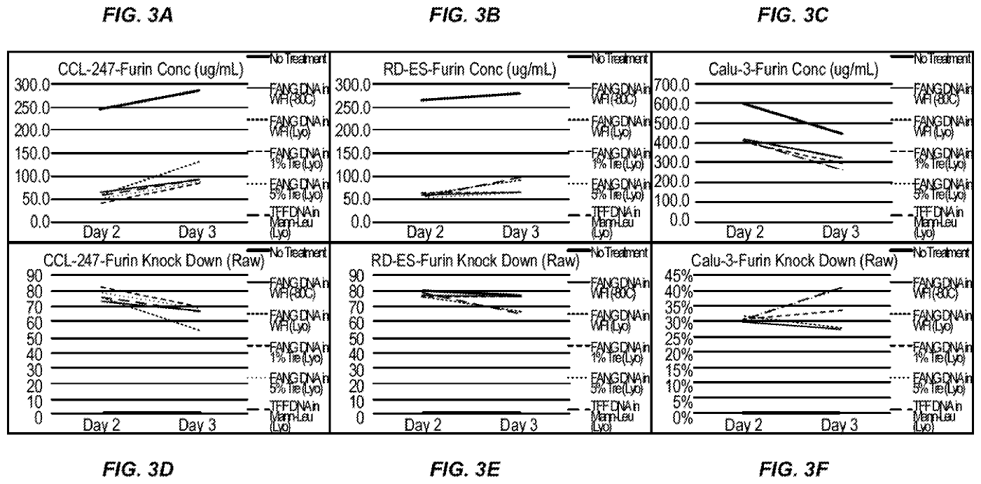

FIGS. 3A-3C show cell culture results of furin concentration using ELISA to measure the concentration in three different types of cell cultures. FIGS. 3D-3F show percent knock down by the plasmid.

FIG. 3C shows the raw furin expression is 2× higher in Calu-3 cells compared to CCL-247 (FIG. 3A) and RD-ES cells FIG. 3B). Furin Knock Down occurred in all 3 cell lines, but 2×less in Calu-3 (FIG. 3E).

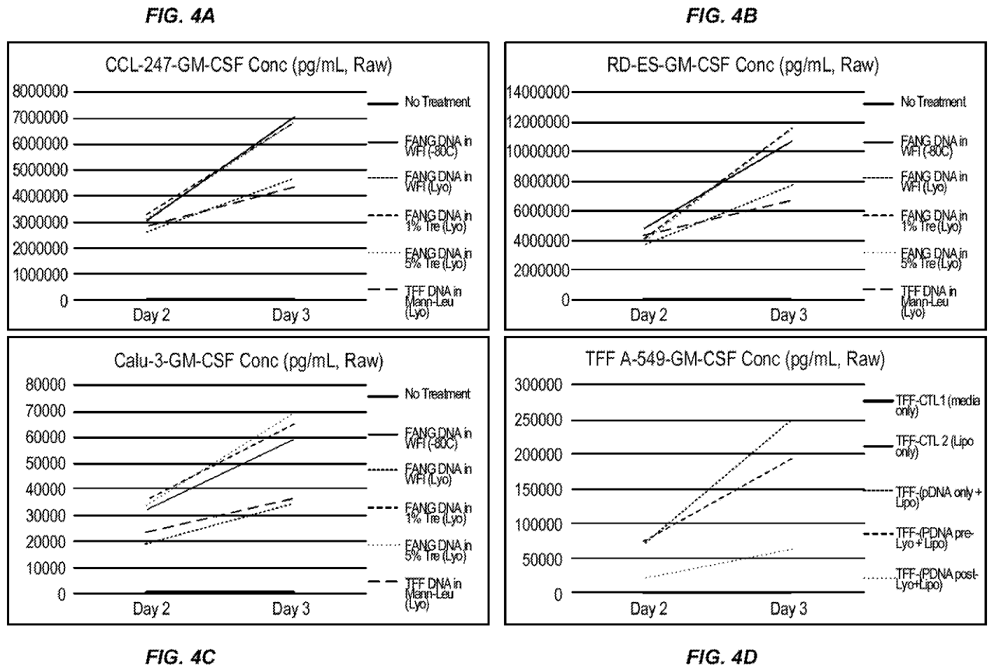

FIGS. 4A-4D show cell culture results of the expression of GM-CSF measured using an enzyme-linked lectin assay (ELLA), in 4 different cell lines. GM-CSF expression was observed in all cell lines, but expression was lower in Calu-3 and A-549 cell lines. A-549 is a human non-small cell lung cancer cell line. A549 is an epithelial carcinoma derived from a 58 year old male patient, known to be KRAS mutant and EGFR wild type.

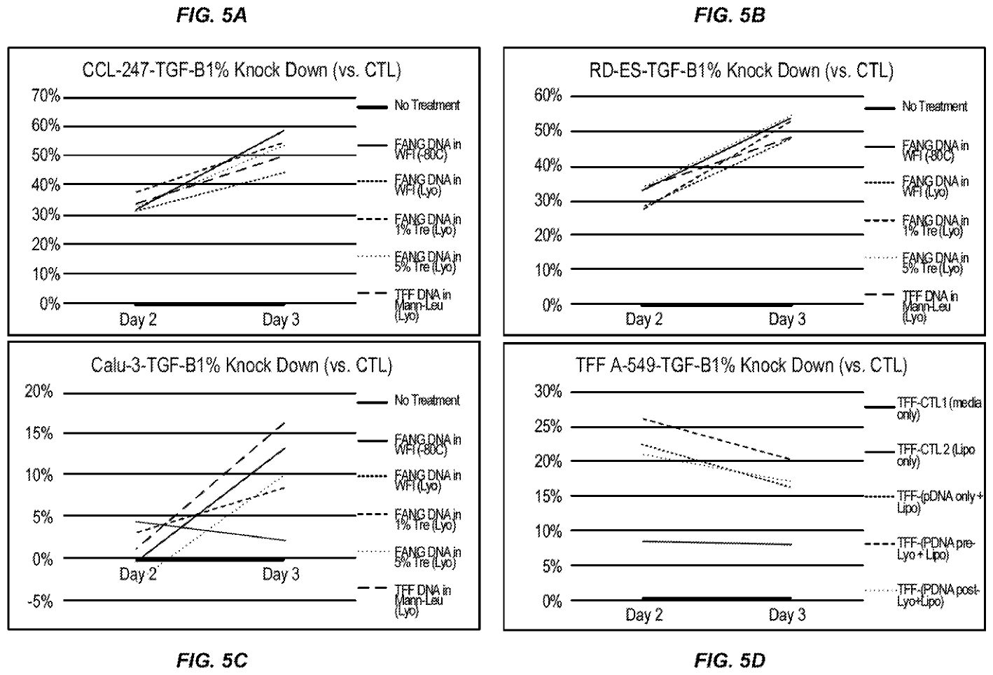

FIGS. 5A-5D show cell culture results of the expression of TGF-β1 measured using an enzyme-linked lectin assay (ELLA), in 4 different cell lines. All cell lines showed TGF-β1 knock-down.

This example clearly demonstrates plasmid activity (GM-CSF expression and TGF-β1 knock down) in 4 different cell lines.

It will be understood that particular embodiments described herein are shown by way of illustration and not as limitations of the disclosure. The principal features of this disclosure can be employed in various embodiments without departing from its scope of the invention. All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

What is claimed is:

1. A method for treating a tumor in the lung of a subject, the method comprising:
administering to the subject an inhalable dosage form, comprising:
a. an expression vector comprising
i. a first insert comprising a nucleic acid sequence encoding a Granulocyte Macrophage Colony Stimulating Factor (GM-CSF) sequence; and
ii. a second insert comprising a nucleic acid sequence encoding at least one bi-functional short hairpin RNA (bi-shRNA) capable of hybridizing to a region of a mRNA transcript encoding furin, thereby inhibiting furin expression via RNA interference wherein the bi-shRNA incorporates cleavage dependent siRNA and cleavage independent miRNA motifs; and
b. at least one stabilizing excipient, to thereby treat the tumor.

2. The method of claim 1, wherein the tumor is caused by a primary lung cancer.

3. The method of claim 1, wherein the primary lung cancer is a member selected from the group consisting of non-small cell lung cancer (NSCLC) and small cell lung cancer (SCLC).

4. The method of claim 1, wherein the tumor is caused by a metastatic event from a cancer in a different part of the subject's body.

5. The method of claim 4, wherein the cancer is a member selected from the group consisting of breast, pancreas, kidney, and skin cancer.

6. The method of claim 4, wherein the cancer is a member selected from the group consisting of brain, bladder, blood, bone, breast, cervical, colorectal, gastrointestinal, endocrine, kidney, liver, lung, ovarian, pancreatic, prostate, and thyroid cancer.

7. The method of claim 1, wherein the second insert of the expression vector comprises a nucleic acid sequence of SEQ ID NO:2 or SEQ ID NO:9.

8. The method of claim 1, wherein the expression vector is a plasmid.

9. The method of claim 1, wherein the inhalable dosage form comprises particles comprising the expression vector and at least one stabilizing excipient selected from the group consisting of glucose, arabinose, maltose, saccharose, dextrose and lactose sucrose, trehalose, human serum albumin (HSA), and glycine.

10. The method of claim 1, wherein the at least one stabilizing excipient is trehalose.

11. The method of claim 9, wherein the particles are from about 0.5 μm to about 10 μm in diameter.

12. The method of claim 9, wherein the particles are from about 1 μm to 3 μm in diameter.

13. The method of claim 1, wherein the expression vector is present from about 1% to 10% by weight.

14. The method of claim 9, wherein the particles are lyophilized particles.

15. The method of claim 1, wherein the GM-CSF is a human GM-CSF sequence.

16. The method of claim 1, wherein the expression vector further comprises a promoter.

17. The method of claim 14, wherein the lyophilized composition is formulated for pulmonary delivery.

18. The method of claim 17, wherein the lyophilized composition is formulated for pulmonary delivery via a device.

19. The method of claim 18, wherein the device is an inhaler or a nebulizer.

20. A method for delivering an expression vector comprising a polynucleotide to the respiratory tract of a subject, the method comprising:
aerosolizing a dry powder formulation of claim, to form a population of aerosolized particles, wherein the aerosolized particles have a diameter related to the diameter of airways in an area of the respiratory tract; and
administering the aerosolized particles to the respiratory tract of the subject, wherein the diameter of the particles targets the particles to the region of the respiratory tract.


SEQUENCE LISTING (9 sequences)

SEQ ID NO: 1 - DNA, 4180 bp, unidentified
  misc_feature 1..4180: note = source = /note="Description of Unknown: mRNA transcript encoding furin sequence"
  source 1..4180: mol_type = unassigned DNA
gcggggaagcagcagcggccaggatgaatcccaggtgctctggagctggatggtgaaggtcggcactcttcaccctcccgagccctgcccgtctcggccccatgcccccaccagtcagccccgggccacaggcagtgagcaggcacctgggagccgaggccctgtgaccaggccaaggagacgggcgctccagggtcccagccacctgtcccccccatggagctgaggccctggttgctatgggtggtagcagcaacaggaaccttggtcctgctagcagctgatgctcagggccagaaggtcttcaccaacacgtgggctgtgcgcatccctggaggcccagcggtggccaacagtgtggcacggaagcatgggttcctcaacctgggccagatcttcggggactattaccacttctggcatcgaggagtgacgaagcggtccctgtcgcctcaccgcccgcggcacagccggctgcagagggagcctcaagtacagtggctggaacagcaggtggcaaagcgacggactaaacgggacgtgtaccaggagcccacagaccccaagtttcctcagcagtggtacctgtctggtgtcactcagcgggacctgaatgtgaaggcggcctgggcgcagggctacacagggcacggcattgtggtctccattctggacgatggcatcgagaagaaccacccggacttggcaggcaattatgatcctggggccagttttgatgtcaatgaccaggaccctgacccccagcctcggtacacacagatgaatgacaacaggcacggcacacggtgtgcgggggaagtggctgcggtggccaacaacggtgtctgtggtgtaggtgtggcctacaacgcccgcattggaggggtgcgcatgctggatggcgaggtgacagatgcagtggaggcacgctcgctgggcctgaaccccaaccacatccacatctacagtgccagctggggccccgaggatgacggcaagacagtggatgggccagcccgcctcgccgaggaggccttcttccgtggggttagccagggccgaggggggctgggctccatctttgtctgggcctcggggaacgggggccgggaacatgacagctgcaactgcgacggctacaccaacagtatctacacgctgtccatcagcagcgccacgcagtttggcaacgtgccgtggtacagcgaggcctgctcgtccacactggccacgacctacagcagtggcaaccagaatgagaagcagatcgtgacgactgacttgcggcagaagtgcacggagtctcacacgggcacctcagcctctgcccccttagcagccggcatcattgctctcaccctggaggccaataagaacctcacatggcgggacatgcaacacctggtggtacagacctcgaagccagcccacctcaatgccaacgactgggccaccaatggtgtgggccggaaagtgagccactcatatggctacgggcttttggacgcaggcgccatggtggccctggcccagaattggaccacagtggccccccagcggaagtgcatcatcgacatcctcaccgagcccaaagacatcgggaaacggctcgaggtgcggaagaccgtgaccgcgtgcctgggcgagcccaaccacatcactcggctggagcacgctcaggcgcggctcaccctgtcctataatcgccgtggcgacctggccatccacctggtcagccccatgggcacccgctccaccctgctggcagccaggccacatgactactccgcagatgggtttaatgactgggccttcatgacaactcattcctgggatgaggatccctctggcgagtgggtcctagagattgaaaacaccagcgaagccaacaactatgggacgctgaccaagttcaccctcgtactctatggcaccgcccctgaggggctgcccgtacctccagaaagcagtggctgcaagaccctcacgtccagtcaggcctgtgtggtgtgcgaggaaggcttctccctgcaccagaagagctgtgtccagcactgccctccagggttcgccccccaagtcctcgatacgcactatagcaccgagaatgacgtggagaccatccgggccagcgtctgcgccccctgccacgcctcatgtgccacatgccaggggccggccctgacagactgcctcagctgccccagccacgcctccttggaccctgtggagcagacttgctcccggcaaagccagagcagccgagagtccccgccacagcagcagccacctcggctgcccccggaggtggaggcggggcaacggctgcgggcagggctgctgccctcacacctgcctgaggtggtggccggcctcagctgcgccttcatcgtgctggtcttcgtcactgtcttcctggtcctgcagctgcgctctggctttagttttcggggggtgaaggtgtacaccatggaccgtggcctcatctcctacaaggggctgccccctgaagcctggcaggaggagtgcccgtctgactcagaagaggacgagggccggggcgagaggaccgcctttatcaaagaccagagcgccctctgatgagcccactgcccaccccctcaagccaatcccctccttgggcactttttaattcaccaaagtatttttttatcttgggactgggtttggaccccagctgggaggcaagaggggtggagactgcttcccatcctaccctcgggcccacctggccacctgaggtgggcccaggaccagctggggcgtggggagggccgtaccccaccctcagcaccccttccatgtggagaaaggagtgaaacctttagggcagcttgccccggccccggccccagccagagttcctgcggagtgaagaggggcagcccttgcttgttgggattcctgacccaggccgcagctcttgcccttccctgtccctctaaagcaataatggtcccatccaggcagtcgggggctggcctaggagatatctgagggaggaggccacctctccaagggcttctgcaccctccaccctgtcccccagctctggtgagtcttggcggcagcagccatcataggaagggaccaaggcaaggcaggtgcctccaggtgtgcacgtggcatgtggcctgtggcctgtgtcccatgacccacccctgtgctccgtgcctccaccaccactggccaccaggctggcgcagccaaggccgaagctctggctgaaccctgtgctggtgtcctgaccaccctcccctctcttgcacccgcctctcccgtcagggcccaagtccctgttttctgagcccgggctgcctgggctgttggcactcacagacctggagcccctgggtgggtggtggggaggggcgctggcccagccggcctctctggcctcccacccgatgctgctttcccctgtggggatctcaggggctgtttgaggatatattttcactttgtgattatttcactttagatgctgatgatttgtttttgtatttttaatgggggtagcagctggactacccacgttctcacacccaccgtccgccctgctcctccctggctgccctggccctgaggtgtgggggctgcagcatgttgctgaggagtgaggaatagttgagccccaagtcctgaagaggcgggccagccaggcgggctcaaggaaagggggtcccagtgggaggggcaggctgacatctgtgtttcaagtggggctcgccatgccgggggttcataggtcactggctctccaagtgccagaggtgggcaggtggtggcactgagcccccccaacactgtgccctggtggagaaagcactgacctgtcatgcccccctcaaacctcctcttctgacgtgccttttgcacccctcccattaggacaatcagtcccctcccatctgggagtccccttttcttttctaccctagccattcctggtacccagccatctgcccaggggtgccccctcctctcccatccccctgccctcgtggccagcccggctggttttgtaagatgctgggttggtgcacagtgatttttttcttgtaatttaaacaggcccagcattgctggttctatttaatggacatgagataatgttagaggttttaaagtgattaaacgtgcagactatgcaaaccag

SEQ ID NO: 2 - RNA, 223 nt, synthetic construct
  misc_feature 1..223: note = source = /note="Description of Artificial Sequence: Synthetic polynucleotide"
  source 1..223: mol_type = other RNA
gatcctgctgttgacagtgagcgcggagaaaggagtgaaaccttagtgaagccacagatgtaaggtttcactcctttctccttgcctactgcctcggaagcagctcactacattactcagctgttgacagtgagcgcggagaaagatatgaaaccttagtgaagccacagatgtaaggtttcactcctttctccttgcctactgcctcggaagcttaataaag

SEQ ID NO: 3 - DNA, 5140 bp, synthetic construct
  misc_feature 1..5140: note = source = /note="Description of Artificial Sequence: Synthetic polynucleotide"
  source 1..5140: mol_type = other DNA
ggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactccaacggtggagggcagtgtagtctgagcagtactcgttgctgccgcgcgcgccaccagacataatagctgacagactaacagactgttcctttccatgggtcttttctgcagtcaccgtcgtcgacggtatcgataagcttgatatcgaattccgctggaggatgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagactatcacctttgaaagtttcaaagagaacctgaaggactttctgcttgtcatcccctttgactgctgggagccagtccaggagtgagaccggccagatgaggctggccaagccggggagctgctctctcatgaaacaagagctagaaactcaggatggtcatcttggagggaccaaggggtgggccacagccatggtgggagtggcctggacctgccctgggccacactgaccctgatacaggcatggcagaagaatgggaatattttatactgacagaaatcagtaatatttatatatttatatttttaaaatatttatttatttatttatttaagttcatattccatatttattcaagatgttttaccgtaataattattattaaaaatatgcttctaaaaaaaaaaaaaaaaaaaaaaacggaattcacgtgggcccggtaccgtatactctagaagatctggcagcggagagggcagaggaagtcttctaacatgcggtgacgtggaggagaatcccggccctaggatgtctagagcggccgcggatcctgctgttgacagtgagcgcggagaaaggagtgaaaccttagtgaagccacagatgtaaggtttcactcctttctccttgcctactgcctcggaagcagctcactacattactcagctgttgacagtgagcgcggagaaagatatgaaaccttagtgaagccacagatgtaaggtttcactcctttctccttgcctactgcctcggaagcttaataaaggatcttttattttcattggatccagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctatt

SEQ ID NO: 4 - DNA, 788 bp, Homo sapiens
  source 1..788: mol_type = genomic DNA
agtacacagagagaaaggctaaagttctctggaggatgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggagtgagaccggccagatgaggctggccaagccggggagctgctctctcatgaaacaagagctagaaactcaggatggtcatcttggagggaccaaggggtgggccacagccatggtgggagtggcctggacctgccctgggccacactgaccctgatacaggcatggcagaagaatgggaatattttatactgacagaaatcagtaatatttatatatttatatttttaaaatatttatttatttatttatttaagttcatattccatatttattcaagatgttttaccgtaataattattattaaaaatatgcttctacttg

SEQ ID NO: 5 - RNA, 15 nt, synthetic construct
  misc_feature 1..15: note = source = /note="Description of Artificial Sequence: Synthetic oligonucleotide"
  source 1..15: mol_type = other RNA
gtgaagccacagatg

SEQ ID NO: 6 - RNA, 21 nt, synthetic construct
  misc_feature 1..21: note = source = /note="Description of Artificial Sequence: Synthetic oligonucleotide"
  source 1..21: mol_type = other RNA
taaggtttcactcctttctcc

SEQ ID NO: 7 - RNA, 21 nt, synthetic construct
  misc_feature 1..21: note = source = /note="Description of Artificial Sequence: Synthetic oligonucleotide"
  source 1..21: mol_type = other RNA
ggagaaaggagtgaaacctta

SEQ ID NO: 8 - RNA, 21 nt, synthetic construct
  misc_feature 1..21: note = source = /note="Description of Artificial Sequence: Synthetic oligonucleotide"
  source 1..21: mol_type = other RNA
ggagaaagatatgaaacctta

SEQ ID NO: 9 - RNA, 245 nt, synthetic construct
  misc_feature 1..245: note = source = /note="Description of Artificial Sequence: Synthetic polynucleotide"
  source 1..245: mol_type = other RNA
gatcctgctgttgacagtgagcgcggagaaaggagtgaaaccttagtgaagccacagatgtaaggtttcactcctttctccttgcctactgcctcggaagcagctcactacattactcagctgttgacagtgagcgcggagaaagatatgaaaccttagtgaagccacagatgtaaggtttcactcctttctccttgcctactgcctcggaagcttaataaaggatcttttattttcattggatc